(19) United States
(12) Patent Application Publication
(10) Pub. No.: US 2004/0082773 A1
(43) Pub. Date: Apr. 29, 2004

(54) Modified PAR receptors, their preparation and their uses for selecting compounds which modulate PAR activity

(75) Inventors: Claire Berthelier, Villebon sur Yvette (FR); Claire Bigogne, Sainte Genevieve des Bois (FR); Stephane Milano, Le Bourg (FR); Emmanuel Normant, Winchester, MA (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/609,831

(22) Filed: Jun. 27, 2003

(30) Foreign Application Priority Data
Jun. 28, 2002 (FR) 02 08173

(51) Int. Cl. C07K 14/705; C07H 21/04

(52) U.S. Cl. 536/23.5; 530/350; 435/69.1; 435/320.1; 435/325

(57) ABSTRACT

The invention describes novel PAR polypeptides and uses thereof. It relates in particular to PAR polypeptides lacking their functional endogenous activating peptide and capable of interacting constitutively with a ligand for a wild-type PAR. The invention also lies in methods of screening for active compounds, using modified PAR polypeptides, and also in the therapeutic use of identified compounds, in particular for the treatment of inflammation, of allergies, of diseases affecting the CNS, of neurodegeneration, or of psychiatric or cardiovascular diseases, and also in methods and compositions which can be used for this purpose. Other subjects of the invention include nucleic acids encoding a modified PAR, vectors and recombinant host cells and transgenic animals containing them, and also their uses in the context of a therapeutic, diagnostic or screening approach, for example.

### FIELD OF THE INVENTION

The present invention relates to novel modified PAR polypeptides and to uses thereof. It relates in particular to PAR polypeptides lacking a functional endogenous activating peptide and capable of interacting with the (or one of the) ligand(s) for a wild-type PAR. The invention also lies in methods of screening for compounds which modulate a PAR receptor, using such modified PAR polypeptides, and also in the therapeutic use of identified compounds, in particular for the treatment of inflammation, of allergies, of diseases affecting the CNS, of neurodegeneration, or of psychiatric or cardiovascular diseases, and also in methods and compositions which can be used for this purpose. Other subjects of the invention include nucleic acids encoding a modified PAR, vectors and recombinant cells containing them, and also their uses in the context of a therapeutic, diagnostic or screening approach, for example.

### BACKGROUND OF THE INVENTION

Approximately 400 types of G protein-coupled receptors (GPCRs) exist, without counting the G protein-coupled olfactory receptors, the number of which is by itself estimated at 400-1000 different receptors.

The activation of a GPCR by its ligand leads to a reorganization of the structure of the receptor, which becomes capable of activating intracellular G proteins. These G proteins in turn activate an entire series of membrane-bound or cytosolic, intracellular effectors (enzymes, ion channels, transporters, etc.). These effectors most of the time enable modulation of the intracellular concentration of secondary messengers such as cyclic adenosine-5-monophosphate (cAMP), inositol triphosphate (IP₃), calcium and diacylglycerols (DAGs), etc.

A new family of receptors, called protease-activated receptors, has recently been described (McFarlane et al. (2001) Pharmacol. Rev. 53:245-282). They are receptors with seven transmembrane domains, coupled to G proteins. The main characteristic of these receptors lies in the fact that they are activated subsequent to a proteolytic cleavage which occurs in the region of their N-terminal end. Such a proteolytic cleavage allows the endogenous peptide contained in the N-terminal region to interact with the active site of the receptor (probably in the region of its second extracellular loop). Before proteolysis, these receptors therefore comprise, in their sequence, a masked endogenous peptide or ligand. After proteolytic cleavage, the N-terminal end comprising the endogenous peptide would thus fold down on the active site of the receptor (FIG. 1), leading to activation thereof. These functional characteristics explain the name of this family of receptors as “protease-activated receptors” (PARs).

### SUMMARY OF THE INVENTION

### The Invention Concerns

A first aspect of the invention relates to a modified PAR polypeptide, characterized in that it lacks its functional endogenous activating peptide and in that it is capable of interacting with (an) exogenous ligand(s) for a wild-type PAR. According to a preferred embodiment, the polypeptides of the invention are PAR polypeptides comprising a deletion of all or part of the N-terminal domain, comprising at least a portion of the sequence of the endogenous activating peptide. The modified PAR polypeptides according to the invention are more preferentially derived from the PAR-1, PAR-2, PAR-3 or PAR-4 receptors, in particular the human receptors.

Another aspect of the invention is a polynucleotide encoding a modified PAR polypeptide (including a functional fragment or variant of the latter) as described above, and also the sequence complementary thereto.

Another aspect of the invention resides in a vector, in particular an expression vector, comprising such a polynucleotide, and also in a recombinant host cell comprising a polynucleotide or a vector according to the invention. The invention also relates to a stable or transient cell line established using such a recombinant host cell. The invention also relates to transgenic (non-human) animals expressing a modified PAR polypeptide and/or comprising a recombinant host cell, a polynucleotide or a vector according to the invention.

Another aspect of the invention relates to a method of producing modified PAR receptors as defined above, or fragments of the latter. The methods are generally based on the expression of a nucleic acid as defined above, in a host cell.

The invention is also directed to a modified PAR polypeptide having an endogenous activating peptide made non functional, and being capable of interacting with an exogenous ligand for a wild-type PAR receptor, wherein the exogenous ligand has an ED50 for the modified PAR polypeptide significantly higher than its ED50 for a wild type PAR receptor. In one embodiment, the exogenous ligand has an ED50 for the modified PAR polypeptide at least 5, 10 or 50 times higher than its ED50 for a wild type PAR receptor.

The invention is also directed to a modified PAR polypeptide wherein a region extending from a first N-terminal residue of the PAR polypeptide up to a residue between the first N-terminal residue of the endogenous activating peptide and a last C-terminal residue of an amino-terminal extracellular domain of the PAR polypeptide is deleted. In one embodiment, the PAR receptor is selected from PAR-1, PAR-2, PAR-3 and PAR-4.

The invention is also directed to a modified PAR polypeptide having a sequence selected from SEQ ID NO:4, sequence SEQ ID NO:5, sequence SEQ ID NO:6, and functional variants and fragments thereof.

The invention is also directed to a polynucleotide encoding a modified PAR polypeptide, where the modified polypeptide has an endogenous activating peptide made non functional, and the modified PAR polypeptide is capable of interacting with an exogenous ligand for a wild-type PAR receptor, wherein the exogenous ligand has an ED50 for the modified PAR polypeptide significantly higher than its ED50 for a wild type PAR receptor.

The invention is also directed to a polynucleotide as set forth in the immediately preceding paragraph which is selected from:

(i) a polynucleotide encoding a polypeptide of SEQ ID NO: 4, 5 or 6;

(ii) a polynucleotide of sequence SEQ ID NO: 1, 2 or 3;

(iii) a polynucleotide hybridizing to a polynucleotide of (i) or (ii) and encoding a modified PAR polypeptide, the modified polypeptide having an endogenous activating peptide made non functional and the modified PAR polypeptide being capable of interacting with an exogenous ligand for a wild-type PAR receptor, wherein the exogenous ligand has an ED50 for said modified PAR polypeptide significantly higher than its ED50 for a wild type PAR receptor;

(iv) a polynucleotide having at least 75% identity with a polynucleotide of (i), (ii) or (iii) and encoding a modified PAR polypeptide as set forth in section (iii) above; and

(v) a polynucleotide encoding a modified PAR polypeptide as set forth in section (iii) above and a sequence of which differ from those of the polynucleotides (i), (ii), (iii) or (iv) due to the degeneracy of the genetic code.

The invention is also directed to expression vectors and recombinant host cells comprising a polynucleotide as set forth above. It is to be understood that this invention comprises those recombinant host cells which express a wild-type PAR receptor and those recombinant host cells which do not express a wild-type PAR receptor. The invention also comprises such recombinant host cells which express a reporter gene for detecting or measuring the activity of the modified PAR polypeptide.

This invention is also directed to methods of preparing the modified PAR polypeptides disclosed herein. In various embodiments, the methods include the steps of:

a) obtaining a polynucleotide encoding a modified PAR polypeptide;

b) inserting the polynucleotide into an expression vector, where the polynucleotide is functionally linked to a promoter; and

c) producing a modified PAR polypeptide from the polynucleotide.

This invention is also directed to methods of screening for, selecting or identifying PAR activity modulator compounds. In various embodiments, the methods include the steps of:

a) bringing a test compound into contact with a modified PAR polypeptide, the modified PAR polypeptide having an endogenous activating peptide which is made non-functional, and the modified PAR polypeptide being capable of interacting with an exogenous ligand for a wild-type PAR receptor; and

b) selecting a compound which binds to said modified PAR polypeptide or determining the activity of the modified PAR polypeptide. The modified PAR polypeptide used in the methods of this invention are capable of interacting with an exogenous ligand for a wild-type PAR receptor, wherein the exogenous ligand has an ED50 for the modified PAR polypeptide significantly higher than its ED50 for a wild type PAR receptor; and/or wherein the exogenous ligand has an ED50 for said modified PAR polypeptide at least 5, 10 or 50 times higher than its ED50 for a wild type PAR receptor; and/or wherein a region extending from a first N-terminal residue of said PAR polypeptide up to a residue between the first N-terminal residue of the endogenous activating peptide and the last C-terminal residue of the amino-terminal extracellular domain is deleted; and/or wherein the PAR receptor is selected from PAR-1, PAR-2, PAR-3 and PAR-4; and/or wherein the modified polypeptide comprises a sequence selected from SEQ ID NO:4, sequence SEQ ID NO:5, sequence SEQ ID NO:6, and functional variants and fragments thereof.

This invention is also directed to such methods which additionally comprise the steps of a) bringing a test compound into contact with said modified PAR polypeptide; and b) selecting or determining the activity of a compound which binds to said modified PAR polypeptide. In some embodiments, determining the activity of compounds which bind to said modified PAR polypeptides may include the steps:

a) providing a recombinant host cell expressing the modified PAR polypeptide in an appropriate culture medium;

b) adding a desired concentration of a test compound in the culture medium;

c) adding a reference ligand in the culture medium obtained at step b);

d) measuring the activity of the modified PAR polypeptide; and

e) comparing the activity of the modified PAR polypeptide obtained at step d) with the activity of the modified PAR polypeptide when step b) is omitted.

In one embodiment, the modified PAR polypeptide activity is determined through a calcium release measurement. It is to be understood that the measurement may be either direct or indirect, but is preferably direct.

This invention is also directed to a method of screening for, selecting or identifying a PAR activity modulator compound, the method further comprising the steps of:

a) providing a recombinant host cell coexpressing the modified PAR polypeptide and a reporter gene for detecting or measuring the activity of the modified PAR polypeptide;

b) adding a desired concentration of a test compound in the culture medium;

c) adding a reference ligand in the culture medium obtained at step b);

d) measuring reporter gene expression; and

e) comparing the reporter gene expression obtained at step d) with the reporter gene expression when step b) is omitted. In one embodiment, the recombinant host cell preferably consists of a CHO cell line. In other embodiments, it is preferred that the reporter gene is a beta lactamase gene, and more preferably that the beta-lactamase gene is placed under the control of a promoter comprising an NFAT domain sensitive to Ca++ ions.

In the methods of this invention, it is also preferred that the modified PAR polypeptide is a modified PAR-2 polypeptide. It is especially preferred that the PAR polypeptide has a sequence selected from SEQ ID NO:4, sequence SEQ ID NO:5, sequence SEQ ID NO: 6, and functional variants and fragments thereof.

In the methods of this invention, it is preferred that the reference ligand is selected from SLIGRL, SLIGKV, SLIGR, propionyl-tc, trans-cinnamoyl-LIGRLO and SFLLR. SLIGRL is a particularly preferred reference ligand.

This invention is also directed to a kit for in vitro screening, selection or identifying of PAR activity modulator compounds, wherein the kit comprises:

a) a polypeptide as defined in claim 1, 2, 3, 4 or 5 or a recombinant host cell as defined in claim 13, 14, 15 or 16;

b) optionally, one or more reagents necessary to perform the PAR polypeptide activity measurement.

Compounds identified by using the modified PAR polypeptides, methods and kits are useful in treating various diseases and conditions such as allergies, tissue repair and healing, inflammatory diseases, including airway inflammation, gastrointestinal tract inflammation, arthritis, asthma, diseases affecting the central nervous system, neurodegeneration, psychiatric diseases, cardiovascular diseases, and in treating diseases and disorders caused by pain transmission such as visceral pain.

### DETAILED DESCRIPTION OF THE INVENTION

The present invention relates to novel modified PAR polypeptides and to uses thereof. It relates in particular to PAR polypeptides lacking a functional endogenous activating peptide and capable of interacting with the (or one of the) ligand(s) for a wild-type PAR. The invention also lies in methods of screening for compounds which modulate a PAR receptor, using such modified PAR polypeptides, and also in the therapeutic use of identified compounds, in particular for the treatment of inflammation, of allergies, of diseases affecting the CNS, of neurodegeneration, or of psychiatric or cardiovascular diseases, and also in methods and compositions which can be used for this purpose. Other subjects of the invention include nucleic acids encoding a modified PAR, vectors and recombinant cells containing them, and also their uses in the context of a therapeutic, diagnostic or screening approach, for example.

Four serine-protease-activated receptors have been identified and characterized. They are the PAR-1, PAR-2, PAR-3 and PAR-4 receptors. These receptors have different cleavage sites, located in the N-terminal domain, and also different endogenous peptides. Thus, PAR-1 and PAR-3 are preferentially activated by thrombin, factor Xa and chymotrypsin, PAR-2 is selectively activated by mastocyte tryptase and trypsin, while PAR-4 is activated both by thrombin and by trypsin. The sequence of the endogenous peptide of these receptors is given below:

| | |
|---|---|
| human PAR-1 | SFLLRN |
| human PAR-2 | SLIGKV |
| murine PAR-2 | SLIGRL |
| human PAR-3 | TFRGAP |
| human PAR-4 | GYPGQV |

The physiological mechanism of activation of these receptors makes it difficult to exploit them, and in particular to search for ligands. Specifically, it makes use of cleavage by proteolysis, and the use, even at a low dose, of proteolytic enzymes is delicate due to their poor specificity. In fact, they can exert their action on all biological material without distinction, thus possibly causing undesired proteolyses, or even, at a high dose, cell lysis.

In order to circumvent this difficulty, it has been proposed to use, in order to activate in vitro the PAR-1, PAR-2 and PAR-4 receptors, exogenous synthetic peptides which are identical or similar in terms of sequence to the endogenous peptides of each receptor, and thus capable of mimicking their action.

Thus, various exogenous synthetic peptides, such as SLIGRL, SLIGKV, SLIGR, trans-cinnamoyl-LIGRLO and SFLLR, have been described and used to artificially activate, independently of proteolytic cleavage, the PAR-2 receptor (Al-Ani et al. (1999) J. Pharmacol. Exp. Ther. 290:753-760).

The main drawback encountered when such exogenous synthetic peptides are used lies in the need to use them at high concentrations in order to obtain activation of the receptor. In the case of PAR-2, for example, 10 to 30 μM of exogenous synthetic peptides are necessary to induce activation of the receptor. The hypothesis put forward to date to explain the need to use these high concentrations is based on the need to compensate the loss of covalent anchoring allowed by the attachment of the endogenous ligand normally generated by proteolytic cleavage. The absence of this anchoring is thus thought to make the binding of the exogenous synthetic peptide to its receptor fragile.

The present application makes it possible to remedy the drawbacks of the prior art indicated above. The present invention in fact provides novel PAR receptors having particular properties which in particular make it possible to develop effective methods of screening for modulators of these receptors.

Very advantageously, the present invention provides PAR receptors which lack their functional endogenous activating peptide and which can be activated effectively in the absence of proteases.

The inventors have in fact, surprisingly, demonstrated that the low affinity of the receptors for the free exogenous synthetic peptides is, at least in part, associated with the poor accessibility of the active site. The three-dimensional representation of the receptor produced by the inventors has made it possible to visualize the stereochemical hindrance of the active site of the receptor when it is in the inactive state, i.e. when the endogenous peptide is not made functional by the action of a protease. Thus, in the non-activated form of the PAR-2 receptor, the portion of the N-terminal domain comprising the endogenous peptide is sufficiently close to the binding site (second extracellular loop) to hinder the passage of a free exogenous peptide and to prevent it from completely reaching the binding site.

The inventors have also shown that the problem of the difficulty of access, and consequently of the use of high concentrations of free exogenous synthetic peptides, can be solved in a particularly advantageous manner by using, according to the invention, a PAR receptor in which the N-terminal portion carrying the endogenous peptide is modified. Specifically, the present invention shows that it is possible to modify the structure of the PAR receptors in such a way as to give them greater affinity for free exogenous synthetic peptides. Such modified PAR receptors are therefore particularly useful for screening for PAR-modulating compounds, and also for studying the properties of these receptors.

A first subject of the invention therefore relates to a modified PAR polypeptide, characterized in that it lacks its functional endogenous activating peptide and in that it is capable of interacting with (an) exogenous ligand(s) for a wild-type PAR. According to a preferred embodiment, the polypeptides of the invention are PAR polypeptides comprising a deletion of all or part of the N-terminal domain, comprising at least a portion of the sequence of the endogenous activating peptide. The modified PAR polypeptides according to the invention are more preferentially derived from the PAR-1, PAR-2, PAR-3 or PAR-4 receptors, in particular the human receptors.

Another subject of the invention is a polynucleotide encoding a modified PAR polypeptide as described above.

Another subject of the invention resides in a vector, in particular an expression vector, comprising such a polynucleotide, and also in a recombinant host cell comprising a polynucleotide or a vector according to the invention. The invention also relates to a stable or transient cell line established using such a recombinant host cell. The invention also relates to transgenic (non-human) animals expressing a modified PAR polypeptide and/or comprising a recombinant host cell, a polynucleotide or a vector according to the invention.

Another subject of the invention relates to a method of producing modified PAR receptors as defined above, or fragments of the latter. The methods are generally based on the expression of a nucleic acid as defined above, in a host cell.

Another subject of the invention is a method of screening, for selecting or identifying active compounds, characterized in that it comprises at least:

a) bringing a test compound into contact with a modified PAR polypeptide according to the invention, and

b) selecting the compound(s) which bind(s) to said polypeptide.

The method is particularly intended for the selection of active compounds which modulate the activity of at least one PAR receptor, the binding to the polypeptide of the invention being characteristic of such a modulation.

The invention also relates to a method of screening, for selecting or identifying active compounds (e.g. compounds which modulate the activity of at least one PAR receptor), characterized in that it comprises at least:

a) bringing a test compound into contact with a modified PAR polypeptide according to the invention, and

b) determining (e.g. detecting) the activity of said modified PAR polypeptide.

In general, the invention concerns the use of a polypeptide or of a nucleic acid molecule as defined above, for selecting compounds which bind to, and/or which modulate the activity of, at least one PAR receptor.

The receptors, vectors, cells, cell lines, transgenic animals and methods of the invention can be used for the identification, selection, characterization or improvement of compounds intended for the treatment of pathological conditions associated with abnormal activity of PAR receptors, more specifically inflammation, allergies, diseases affecting the central nervous system (CNS), neurodegeneration, or psychiatric or cardiovascular diseases.

For example, PAR-2 plays important roles as a mediator of tissue healing and repair, nociception, and neurogenic inflammation. PAR-2 may also have a potential role in mediating non-neurogenic inflammatory responses. As a G protein-coupled receptor that is rapidly desensitized following activation, PAR-2 is generally considered to mediate acute inflammatory responses. However, a recent report demonstrated that PAR-2 mediates also chronic inflammatory processes such as rheumatoid arthritis (Schmidlin et al., November 2002, the journal of immunology, 5316-5321 ; Coughlin et al., 2003, J. Clin. Invest., 111 :25-27 ; Vergnolle et al., 2001, TRENDS in pharmacological sciences, 22(3) :146-152).

The invention also relates to the use of a compound identified using a method according to the invention, for producing a composition intended to modulate the activity of PAR receptors in vivo, in particular for the treatment of inflammation, of allergies, of diseases affecting the CNS, of neurodegeneration, of psychiatric or cardiovascular diseases.

### Definitions

In the present description, the expression “endogenous activating peptide” should be understood to mean any peptide included in the structure of a wild-type PAR and capable of binding, after enzymatic cleavage, to the active site (activation site) of said PAR.

The expression “ligand for a wild-type PAR” is understood to mean any molecule of endogenous or exogenous origin capable of binding to the active site of a wild-type PAR receptor, preferably selectively. This term includes in particular the endogenous activating peptides and also exogenous peptides. Such exogenous peptides are not included in the sequence of the PAR receptor to which they bind, i.e. are not endogenous, but are added during the PAR receptor binding and/or activity assay. They are generally synthetic peptides, i.e. peptides produced artificially, the sequence of which is (or comprises a region) identical or similar to that of an endogenous activating peptide. Preferably, this term denotes exogenous peptides capable of binding to the active site of a wild-type PAR receptor.

The term “reference ligand” or “ligand of reference” is intended to mean a ligand that binds to, or binds to and activates a modified PAR polypeptide of the invention, or a wild type PAR receptor respectively. Preferentially the “reference ligand” binds to, or binds to and activates a wild type PAR receptor. The reference ligand may be an exogenous synthetic peptide, an antibody, etc. When the modified PAR polypeptide is a PAR-2 polypeptide, the reference ligand is preferably SLIGRL, SLIGKV, SLIGR, propionyl-tc, trans-cinnamoyl-LIGRLO or SFLLR.

The term “PAR polypeptide” is intended to mean any polypeptide, preferably derived from mammals, more preferentially from humans or from rodents, and also its variants, analogues, orthologues, homologues and derivatives, and also its fragments, which is a receptor of the GPCR family and which can be activated by a protease, allowing an endogenous ligand located in its N-terminal end to interact with its active site.

Illustrative but nonlimiting examples of amino acid sequence of PAR receptors have been published and/or are accessible on the sequence databases such as Genseq, Swissprot, Genbank, Embl or PIR.

The expressions “polynucleotides having at least x% identity with a reference polynucleotide” and “polypeptides having at least x% identity with a reference polypeptide” are intended to mean polynucleotides or polypeptides, respectively, in which the sequence of residues (nucleotides or amino acids respectively) shows a percentage identity, as defined below, equal to or greater than x.

The percentage identity of a polynucleotide or polypeptide sequence, compared to another polynucleotide or polypeptide sequence, is determined after comparison of these two optimally aligned sequences over a window of comparison. Insertions and/or deletions of residues can be introduced into one or other of the two aligned sequences in this window of comparison, in order to optimize the alignment between the two sequences. The window of comparison then comprises a certain number of positions, the total number of positions corresponding to the size of the window. Each position of the window can have one of the following three configurations:

1°/ There is a residue (nucleotide or amino acid) at this position on the first sequence of the alignment and there is a different residue (nucleotide or amino acid) at this same position on the second sequence of the alignment, in other words the second sequence of the alignment has a substitution at this position compared to the first sequence.

2°/ There is a residue (nucleotide or amino acid) at this position on the first sequence of the alignment and there is the same residue (nucleotide or amino acid) at this same position on the second sequence of the alignment.

3°/ There is a residue (nucleotide or amino acid) at this position on the first sequence of the alignment and there is no residue (nucleotide or amino acid) at this same position on the second sequence, in other words the second sequence of the alignment has a deletion at this position compared to the first sequence.

The number of positions, in the window of comparison, belonging to category 1°/ defined above is referred to as R1.

The number of positions, in the window of comparison, belonging to category 2°/ defined above is referred to as R2.

The number of positions, in the window of comparison, belonging to category 3°/ defined above is referred to as R3.

The percentage identity (% id) may in particular be calculated in one of the following ways:

% id=R2/(R1+R2+R3)×100,

% id=(R2+R3)/(R1+R2+R3)×100,

% id=R2/total length of the reference sequence×100, or

% id=(R2+R3)/total length of the reference sequence×100.

In a particular embodiment, the percentage identity may be calculated in the following way: % id=R2/total length of the reference sequence×100.

In another particular embodiment, the percentage identity may be calculated in the following way: % id=(R2+R3)/total length of the reference sequence×100.

The alignment of the sequences to be compared may be produced using a variety of sequence comparison algorithms and programs known to those skilled in the art. Nonlimiting examples of such sequence comparison algorithms/programs are TBLASTN, BLASTP, FASTA, TFASTA, FASTDB and WU-BLAST (Pearson and Lipman; Karlin and Altschul; Altschul et al. 1990 and 1993 and 1997; Thompson et al.; Higgins et al.; Brutlag et al.), but also Gapped-BLAST, and PSI-BLAST.

In a particular embodiment, the BLAST programs (in particular BLASTP, BLAST3, BLASTN, BLASTX, TBLASTN, TBLASTX) are used with their default parameters or with parameters specified by the user. BLAST is preferentially used with the BLOSUM62 comparison matrix (Gonnet et al.; Henikoff and Henikoff). However, the PAM and PAM250 matrices can also be used (Schwartz and Dayoff).

In another embodiment of the invention, the FASTDB program is used with the following parameters for DNA sequences (an RNA sequence may also be used by converting the Us to Ts beforehand): Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the object sequence if it is shorter. In another embodiment of the invention, the FASTDB program is used with the following parameters for protein sequences: Matrix=PAM, k-tuple=2, Mismatch Penalty=1, Joining Penalty=20, Randomization Group Length=0, Cutoff Score=1, Gap Penalty=5, Gap Size Penalty=0.05, Window Size=500 or the length of the object sequence if it is shorter.

In another embodiment of the invention, the Smith-Waterman method is used with the PAM or PAM250 comparison matrices or preferentially the BLOSUM matrices such as BlOSUM60 or BLOSUM62, and with the default parameters (Gap Opening Penalty=10 and Gap Extension Penalty=1) or with parameters specified by the user, greater than these default values.

In the present application the expression “active compounds” should be understood to mean compounds which modulate the activity of at least one PAR receptor. Therefore synonyms of the meaning of an “active compound” in the present application are “a PAR polypeptide modulator”, “a PAR polypeptide modulator compound”, “a PAR modulator”, and “a PAR modulator compound”.

In the present application the expression “test compounds” should be understood to mean a compound not yet known to modulate the activity of any PAR receptor. The expression “compound which modulates” should be understood to mean any compound capable of activating or inhibiting a PAR receptor, in particular any agonist or antagonist of a PAR receptor. These are preferentially selective modulators, i.e. modulators having no significant direct action on another particular membrane receptor. The test compounds can be peptides, nucleic acids, organic or inorganic chemical molecules, lipids, saccharides, collections of chemical compounds.

In the present application the expression “PAR precursor” or “precursor” should be understood to mean a wild type(WT) PAR polypeptide with a native signal peptide. Accordingly, in the present application the expression “mature PAR polypeptide”, “mature polypeptide”, “mature wild type (WT) PAR”, and “mature PAR protein” should be understood to mean a wild type PAR obtained after signal peptide cleavage.

In the present application, the expression “PAR”, “PAR polypeptide”, and “PAR receptor” should be understood to be synonyms.

In the present application, the expression “ED₅₀” or “ED50” is considered to be the concentration of ligand necessary to achieve 50% of the maximum PAR activity. In the context of the invention the fact that “the exogenous ligand has a significantly higher (better) affinity for the modified PAR polypeptide of the invention in comparison to its affinity for a wild type (WT) PAR receptor” is considered to mean that said exogenous ligand has an ED50 for said modified PAR polypeptide that is significantly higher than its ED50 for a wild type PAR receptor.

In the context of the invention a significantly higher ED50 is intended to mean an ED50 at least 5, 10 or 50 times higher.

### Modified PAR Polypeptide

The wild-type PAR polypeptides are generally described as structures with seven transmembrane alpha-helices. The amino-terminal end of the polypeptide is extracellular and comprises the sequence of the endogenous peptide. The carboxy-terminal end is intracellular (cf.: FIG. 1). Three loops are extracellular and the other three are intracellular. These polypeptides are subject to post-translational modifications, such as N-glycosylation, acylation with lipid compounds (sometimes forming a pseudo-fourth intracellular loop), formation of disulphur bridges between the side chains of two cysteine residues, etc. These receptors are involved in diverse biological and physiological processes, the dysfunction of which is associated with various pathological conditions.

A first aspect of the invention resides in the demonstration that it is possible to modify the structure of PAR receptors in order to impart to them improved properties of activation and of binding to ligands. More particularly, a subject of the present invention is modified PAR polypeptides, characterized in that they lack a functional endogenous activating peptide and in that they are capable of interacting with an exogenous ligand for a wild-type PAR.

In the context of the present invention, the extracellular amino-terminal domain containing the endogenous peptide undergoes a modification. A PAR polypeptide according to the invention is more particularly characterized in that the endogenous activating peptide is made nonfunctional by modification of all or part of the N-terminal extracellular domain of said polypeptide. The modification may comprise a deletion and/or a mutation of one or more residues, or several deletions and/or mutations. The term “deletion” should be understood to mean the loss of one or more contiguous amino acids. The term “mutation” is typically intended to mean a point mutation, i.e. an addition or a substitution of an amino acid.

In a preferred embodiment of the invention, the modification comprises a deletion of all or part of the N-terminal extracellular domain comprising at least a portion of the sequence of the endogenous activating peptide. In a particularly preferred embodiment, the deletion concerns a region which extends from the first (N-terminal) residue of the mature PAR polypeptide up to a residue between the first (N-terminal) residue of the endogenous activating peptide and the last (C-terminal) residue of the N-terminal extracellular domain located upstream of the first transmembrane domain. It should be noted that the position of the transmembrane domains may be slightly different depending on the predictive algorithms used and can therefore vary by a few amino acids relative to the real position of the domains, and from one prediction to the other.

Modified PAR polypeptides which are particularly preferred according to the invention completely lack an endogenous activating peptide, i.e. comprise a deletion covering all of the sequence of said endogenous peptide. They are advantageously characterized by a deletion of the N-terminal region extending at least from the first to the last residue of the endogenous peptide. Consequently, another particular subject of the invention resides in a modified PAR receptor comprising the deletion of a region which extends from the first residue of the mature polypeptide up to a residue between the last residue of the endogenous activating peptide and the last residue of the amino-terminal extracellular domain. Another particular subject of the invention resides in a modified PAR receptor characterized by a deletion extending from the first residue of the mature polypeptide up to the last residue of the endogenous activating peptide.

The present application shows that such deletions do not affect the functionality of the receptor, but improve the affinity of exogenous ligands and make it possible to be free of the use of proteases. Such receptors are therefore particularly suitable for screening for compounds which modulate the activity of PAR receptors.

According to a particular embodiment of the invention, the modified PAR receptor is a mammalian, preferably human PAR receptor. Preferentially, the PAR receptor is chosen from PAR-1, PAR-2, PAR-3 and PAR-4 or any other receptor which can be activated by a protease, preferably PAR-2.

The PAR-1 receptor, also called thrombin receptor or coagulation factor II receptor, has been identified in humans (Vu et al. (1991) Cell 64:1057-1068; Bahou et al. (1993) Blood 82:1532-1537), in monkeys (Swissprot P56488) and also in various rodents (Rasmussen et al. (1991) FEBS Lett. 288:123-128; Kahn et al. (1996) Mol. Med. 2:349-357; Xue et al. (1996) Genome 7:625-626, Swissprot P30558; Zhong et al. (1992) J. Biol. Chem. 267:16975-16979). The amino acid sequence of the precursor of the human PAR-1 receptor is available on Swissprot (accession no: P25116). In this Swissprot entry, the endogenous ligand for PAR-1 corresponds to amino acids 42-47 of the precursor, and the first transmembrane (TM) domain is predicted to start at residue 103.

A particular subject of the invention resides in a modified human PAR-1 receptor comprising the deletion of a region which extends from the first residue of the mature polypeptide up to a residue between residues 42 and 102 inclusive of the precursor. Another particular subject of the invention resides in a modified PAR-1 receptor comprising the deletion of a region which extends from the first residue of the mature polypeptide up to a residue between residues 47 and 102 inclusive of the precursor. Another particular subject of the invention resides in a modified PAR-1 receptor characterized by a deletion extending from residues 1 of the mature polypeptide to 47 inclusive of the precursor.

The PAR-2 receptor has been identified in humans (Nystedt et al. (1995) Eur. J. Biochem. 232:84-89; Boehm et al. (1996) Biochem J. 314:1009-1016; Kahn et al. (1996) above) and also in rats (Saifeddine et al. (1996), Br. J. Pharmacol. 118:521-530) and mice (Nysted et al. (1995) J. Biol. Chem. 270:5950-5955). The amino acid sequence of the precursor of PAR-2 is available on Swissprot (accession no.: P55085). In this Swissprot entry, the endogenous ligand for PAR-2 corresponds to amino acids 37-42 of the precursor, and the first transmembrane (TM) domain is predicted, by the inventors, to start at amino acid 80.

A preferred subject of the invention resides in a modified human PAR-2 receptor comprising the deletion of a region which extends from the first residue of the mature polypeptide up to a residue between residues 37 and 79 inclusive of the precursor. Another particularly preferred subject of the invention resides in a modified human PAR-2 receptor comprising the deletion of a region which extends from the first residue of the mature polypeptide up to a residue between residues 42 and 79 inclusive of the precursor. Specific examples of implementation of the invention are PAR-2 polypeptides comprising a deletion of residues 1-42 (Par2Mut-2); 1-56 (Par2-Mut3) or 1-77 (Par-2Mut4), which are described in FIG. 2; the C terminal end of the deletion being calculated according to the precursor sequence numbering. In this regard, the invention resides in particular in modified human PAR-2 polypeptides comprising a sequence selected from the group consisting of sequence SEQ ID NO:4 (PAR-2Mut-2), sequence SEQ ID NO:5 (PAR-2Mut-3) and sequence SEQ ID NO:6 (PAR-2Mut-4). These polypeptides are encoded by the nucleic acid sequences of SEQ ID Nos.: 1 to 3 respectively.

In a more preferred embodiment of this invention, a modified human PAR-2 polypeptide comprises a sequence selected from the group consisting of sequence SEQ ID NO:4 (PAR-2Mut-2) and sequence SEQ ID NO:5 (PAR-2Mut-3). In a most preferred embodiment of the present invention, a modified human PAR-2 polypeptide has a sequence selected from the group consisting of sequence SEQ ID NO:4 (PAR-2Mut-2) and sequence SEQ ID NO:5 (PAR-2Mut-3).

The PAR-3 receptor has been identified in humans and mice (Ishihara et al. (1997) Nature 386:502-506). The amino acid sequence of the precursor of human PAR-3 is available on Swissprot (accession no.: 000254). In this Swissprot entry, the endogenous ligand for PAR-3 corresponds to amino acids 39-44 of the precursor, and the first transmembrane (TM) domain is predicted to start at amino acid 95.

Another particular subject of the invention resides in a modified human PAR-3 receptor comprising the deletion of a region which extends from the first residue of the mature polypeptide up to a residue between residues 39 and 94 inclusive of the precursor. Another particular subject of the invention resides in a modified human PAR-3 receptor comprising the deletion of a region which extends from the first residue of the mature polypeptide up to a residue between residues 44 and 94 inclusive of the precursor. Another particular subject of the invention resides in a modified human PAR-3 receptor characterized by a deletion extending from residues 1 of the mature polypeptide to 44 inclusive of the precursor.

The PAR-4 receptor has been identified in humans (Xu et al. (1998) Proc. Natl. Acad. Sci. USA 95:6642-6646; Kahn et al. (1998) Nature 394:690-694; Nakanishi-Matsui et al. (2000) Nature 404:609-613) and also in mice (Kahn et al. (1998) J. Biol. Chem. 273(36):23290-6 and Nature 394(6694) :690-4). The amino acid sequence of the precursor of human PAR-4 is available on RefSeq (accession no.: NP_{—}003941). In this entry, the endogenous ligand for PAR-4 corresponds to amino acids 48-53 of the precursor, and the first transmembrane (TM) domain is predicted to start at amino acid 79.

Another particular object of the invention resides in a modified human PAR-4 receptor comprising the deletion of a region which extends from the first residue of the mature polypeptide up to a residue between residues 48 and 78 inclusive of the precursor. Another particular subject of the invention resides in a modified human PAR-4 receptor comprising the deletion of a region which extends from the first residue of the mature polypeptide up to a residue between residues 53 and 78 inclusive of the precursor. Another particular subject of the invention resides in a modified human PAR-4 receptor characterized by a deletion extending from residues 1 of the mature polypeptide to 53 inclusive of the precursor.

According to another embodiment of the invention, the PAR polypeptides of the invention comprise one or more mutations. The mutation(s) may also be combined with the deletion(s)

Whatever the modification(s), it (they) has (have) the effect of making the endogenous peptide nonfunctional, i.e. incapable of binding to the active site of the receptor and of interfering significantly with the binding of an exogenous ligand. Such a nonfunctional endogenous peptide is therefore unable of playing its physiological role of activator of the PAR receptor which bears it.

A particularly advantageous characteristic of the receptors of the invention resides in the fact that they are capable of binding to ligands for PAR receptors, including exogenous synthetic peptides, with better affinity. The active site of the modified receptor according to the invention can thus be activated by ligands without it being necessary to use a protease or very high concentrations of exogenous ligand.

The invention also relates to any fragment of a modified PAR receptor as defined above. The term “fragment” generally denotes a polypeptide comprising at least five consecutive residues of the sequence of a modified PAR polypeptide, preferably at least 8 to 10 amino acids, even more preferentially at least 12, 15, 20, 25, 30, 40, 50, 75, 100, 150, 200, 250, 350 or 400 amino acids. Fragments of the invention are advantageously fragments which conserve the ability to interact with an exogenous ligand for a wild-type PAR. Such fragments can be produced by enzymatic, chemical or physical cleavage and/or by recombinant or chemical synthesis.

The invention also relates to any functional variant of a modified PAR polypeptide as defined above. The expression “functional variant of a modified PAR polypeptide according to the invention” should be understood to mean, according to the invention, any polypeptide having a sequence which is different (mutation, deletion, etc.) from that of said modified PAR polypeptide, which does not comprise a functional endogenous activating ligand, and which is capable of interacting with an exogenous ligand for a wild-type PAR. They may in particular be polypeptides exhibiting one or more polymorphisms, polypeptides originating from various products of splicing, homologues derived from different species, etc. They may also be PAR polypeptides according to the invention, comprising one or more additional deletions and/or mutations in regions which do not significantly affect the binding of a ligand to the active site (for example in an intracytoplasmic domain; C-terminal domain, etc.). Preferentially, the functional variants are at least 40%, 50%, 60%, 70%, 80%, 85%, 90%, 95%, 96%, 97%, 98% or 99% identical to a reference modified PAR polypeptide. The modified PAR polypeptides according to the invention, and their functional variants, may comprise additional residues, such as targeting elements (e.g. signal peptide), or labels (e.g. a tag for identifying the cell or the membrane fragment in which the receptor is expressed).

The modified PAR polypeptides according to the invention, and their functional variants, can be further modified during or after translation, for example by glycosylation, acetylation, phosphorylation, amidation, addition of blocking or protective groups, proteolytic cleavage, attachment to an antibody or to another cellular ligand, attachment to a chemical group to increase stability, attachment to a labelled molecule to allow detection and isolation of the polypeptide, etc.

The polypeptides of the invention can be produced by any technique known to those skilled in the art, and in particular by the recombinant pathway, by chemical synthesis, by enzymatic digestion or by combinations of these methods.

### Nucleic Acids and Expression Vectors

Another subject of the invention concerns a polynucleotide encoding a modified PAR polypeptide (including a functional fragment or variant of the latter) as described above, and also the sequence complementary thereto. Such a polynucleotide may be a DNA or an RNA, more particularly a genomic DNA (gDNA), a complementary DNA (cDNA), a messenger RNA (mRNA), etc.

Typically, the polynucleotide is a cDNA. A polynucleotide according to the invention can be obtained and prepared in various ways known to those skilled in the art. Thus, the nucleic acid sequences encoding various PAR receptors are available on diverse sequence databanks. Their accession numbers are in particular RefSeq NM_{—}001992 (PAR1); RefSeq NM_{—}005242 (PAR2); RefSeq NM_{—}004101 (PAR3); RefSeq NM_{—}003950 (PAR4). These sequences have also been published (see references of the PAR-1 to 4 polypeptides stated above) The polynucleotides of the invention can be prepared from the available nucleic acid sequences encoding wild-type PAR receptors, by various techniques known to those skilled in the art, including the techniques of chemical synthesis, enzymatic digestion, mutagenesis, hybridization, PCR amplification, etc. A particular example of production of a polynucleotide according to the invention, using the mutagenesis of a polynucleotide encoding a wild-type PAR-2, is described in detail in the examples of implementation of the present application.

According to a particular embodiment, the invention relates to a polynucleotide chosen from:

(i) the polynucleotides encoding modified PAR polypeptides according to the invention, preferably the polypeptides of SEQ ID NO: 4, 5 or 6;

(ii) the polynucleotides of sequence SEQ ID NO: 1, 2 or 3;

(iii) the polynucleotides hybridizing to a polynucleotide according to (i) or (ii), preferably under highly stringent conditions, and encoding a modified PAR polypeptide according to the invention;

(iv) the polynucleotides having at least 60, 65, 70, 75, 80, 85, 90, 95, 97, 98, 99% identity with a polynucleotide according to (i), (ii) or (iii) and encoding a modified PAR polypeptide according to the invention;

(v) the polynucleotides encoding a modified PAR polypeptide according to the invention and the sequences of which differ from those of the polynucleotides (i), (ii), (iii) or (iv) due to the degeneracy of the genetic code.

The techniques for hybridizing cDNA clones or genomic clones are well known to those skilled in the art (see, for example, Sambrook and Russell, (2001) Molecular cloning: A laboratory manual, Cold Spring Harbor Laboratory Press, New York, 6:33-6:58 and 7:21-7:45). Briefly, a polynucleotide encoding a modified PAR polypeptide according to the invention, or one of its fragments, can be used as a probe to identify polynucleotides exhibiting a more or less high degree of identity with said probe, by varying the hybridization stringency conditions. In the context of the invention, highly stringent conditions, i.e. allowing the hybridization of polynucleotides having a high degree of identity with the probe, are preferable. Many hybridization conditions known to those skilled in the art can be used, in which the high stringency can be obtained with a high hybridization and/or washing temperature, a high ionic strength of the buffer, and/or the presence of formamide in the hybridization and/or washing buffer, etc. By way of example, the conditions below are considered to be highly stringent for a nucleic acid molecule of approximately 20 nucleotides:

prehybridization carried out at 65° C. in a 6×SSC buffer, 5× Denhardt's solution, 0.5% SDS and 100 μg/ml of salmon sperm DNA in order to block nonspecific sites,

hybridization for 12 to 16 hours in the same buffer, also comprising the probe which would preferably be labelled, for example radioactively,

2 washes of 5 minutes, preferably at 65° C. in a 2×SSC buffer and 0.1% SDS

1 wash of 30 minutes, preferably at 65° C. in a 2×SSC buffer and 0.1% SDS

1 wash of 10 minutes, preferably at 65° C. in a 0.1×SSC buffer and 0.1% SDS.

Of course, these conditions should be adjusted as a function of the length of the probe used.

The invention also encompasses a recombinant vector, for example a cloning or expression vector, comprising a polynucleotide encoding a modified PAR polypeptide as described above. The vector which can be used in the context of the present invention may be a plasmid, a virus, a phage, an artificial chromosome, etc.

Particular expression plasmids which can be used in the invention are mentioned below, only by way of examples: pQE70, pQE60, pQE-9 (Quiagen), pD10, phagescript, psiX174, p.Bluescript SK, pNH8A, pNH16A, pNH18A, pNH46A (Stratagene); pKK223-3, pKK233-3, pDR540, pRIT5 (Pharmacia); PWLNEO, pSV2CAT, pOG44, pXT1, pSG (Stratagene); pSVK3, pBPV, pMSG, pSVL (Pharmacia); pQE-30 (QIA express).

Other recombinant vectors which can be used are the P1 bacteriophages, baculoviruses (able to propagate in insect cell lines or cells) and in particular the baculovirus pVL used to transfect the SF9 cell line (ATCC No. CRL 1711).

Expression vectors according to the invention can also originate from an adenovirus. A preferred recombinant adenovirus according to the invention is the human adenovirus type 2 or 5 (Ad 2 or Ad 5) or an adenovirus of animal origin (applications WO 95/02697 and WO 94/26914).

Retroviruses suitable for gene transfer in vitro or ex vivo can also be used, particularly retroviruses chosen from the Moloney virus (MoMuLV), the murine sarcoma virus, the Rous sarcoma virus and lentiviruses (HIV, etc.).

Another viral vector which can be used is the AAV virus.

For a use in the context of the present invention, the virus-derived vectors are generally replication-defective viruses. They can be produced by any technique known to those skilled in the art, for example using encapsidation lines.

A particular subject of the invention concerns an expression vector comprising a polynucleotide encoding a PAR polypeptide according to the invention (including a functional variant or fragment of the latter), said nucleic acid molecule being functionally linked to a promoter sequence.

The promoter sequence (or the promoter) may be of viral, cellular or synthetic origin. It may be a promoter which acts constitutively or in a regulated manner. This promoter may be strong or weak, ubiquitous or else specific for one or more tissues, etc. Suitable promoters can be chosen in particular from the SV40, Rous sarcoma virus (RSV), thymidine kinase (TK) or cytomegalovirus (CMV) viral promoters, from eukaryotic promoters such as the, tetracyclin promoter, and from bacterial promoters such as the pLac, pTrp, T7, etc. promoters. Preferred promoters are the CMV or SV40 promoters. It is understood that the invention is not limited to the choice of a particular promoter, and also includes the use of cellular promoters, in particular promoters of housekeeping or strongly expressed genes.

The vector may comprise other regulatory sequences (e.g. sequences for secretion or for targeting to membranes, transcription termination sequence, origin of replication, etc.) and/or a marker gene (e.g. the product of expression of which makes it possible to identify the cells comprising the vector).

### Host Cells and Animals Expressing a Modified PAR Polypeptide

Another subject of the present invention concerns any recombinant or genetically modified host cell which expresses a modified PAR polypeptide as defined above. The expression “recombinant host cell” is understood to mean any host cell comprising a polynucleotide or a vector as described above, in particular a recombinant expression vector.

The host cells can be of varied nature and origin, in particular prokaryotic or eukaryotic. Preferred host cells according to the invention are chosen from a) prokaryotic host cells originating, for example, from strains of Escherichia coli (e.g. strain DH5-α), of Bacillus subtilis, of Salmonella typhimurium, of Pseudomonas, of Streptomyces or of Staphylococcus; b) eukaryotic animal host cells, in particular mammalian cells, insect cells, amphibian cells (oocytes for example); yeast cells; plant cells, etc. Among eukaryotic cells, it is thus possible to use, in the context of the present invention, the following cells: T cell lines (ECACC U937, 85011440; ECACC J. CaM1.6, 96060401; ECACC Jurkat E6.1, 88042803 and ECACC J45.01, 93031145), Hela cells (ATCC No. CCL2; No. CCL2.1; No. CCL2.2), Cv 1 cells (ATCC No. CCL70), COS cells (ATCC No. CRL 1650; No. CRL 1651), Sf-9 cells (ATCC No. CRL 1711), C127 cells (ATCC No. CRL-1804), 3T3 cells (ATCC No. CRL-6361), CHO cells (ATCC No. CCL-61), human kidney HEK 293 cells (ATCC No. 45504; No. CRL-1573), BHK (ECACC No. 84100 501; No. 84111301), PC12 (ATCC No. CRL-1721), NT2, SHSY5Y (ATCC No. CRL-2266), NG108 (ECACC No. 88112302) and F11, SK-N-SH (ATCC No. CRL-HTB-11), SK-N-BE(2) (ATCC No. CRL-2271), IMR-32 (ATCC No. CCL-127), KNRK cells (Al-Ani et al., 1999, J Pharmacol exp therap, Vol. 290, Issue 2, 753-760).

The nucleic acid of interest can be introduced into the host cell by any means known to those skilled in the art, and in particular by transfection, electroporation, microinjection, infection using a viral vector or a bacteriophage containing the sequence(s) of interest, by cell fusion or gene transfer mediated by a chromosome or a microcell, by spheroplast fusion, etc.

Preferred cell lines or populations are those which allow the expression of large amounts of PAR or fragments of the latter.

In a preferred embodiment of the invention, host cells are used which do not naturally express the wild-type PAR receptor corresponding to the modified PAR receptor according to the invention. A preferred cell system in which a nucleic acid molecule according to the invention can be expressed comprises bacterial or mammalian cells, in particular the KNRK, CHO, COS or HEK lines.

Most preferred host cells according to the invention are CHO cells.

The invention also relates to an established cell line, i.e. a cell population in which the cells are homogeneous compared to one another, said cell line being stably or transiently transfected with a nucleic acid or a vector as defined above. Preferably, the line does not express any endogenous wild-type PAR receptor.

Another particular subject of the invention concerns a cell or a cell line as defined above, also comprising a reporter system for detecting or measuring the activity of the PAR receptor.

The reporter system generally comprises a reporter gene the expression of which can be detected, observed or measured by any technique (auxotrophy, fluorescence, luminescence, resistance, etc.), and is under the control of regulatory regions the activity of which can be modulated, directly or indirectly, by the activity of a PAR receptor.

Therefore, in the context of the invention a reporter gene allows to detect or measure the activity of a modified PAR receptor of the invention.

The reporter gene may, for example, be any nucleic acid encoding luciferase (e.g. from firefly or from Renilla), secreted alkaline phosphatase, β-galactosidase, lactamase, chloramphenicol acetyl transferase (CAT), human growth hormone (hGH) or β-glucuronidase (Gluc), etc. Preferred reporter gene are nucleic acids encoding luciferase (e.g. from firefly or from Renilla), β-galactosidase, lactamase, acetyl transferase (CAT),or β-glucuronidase (Gluc).

Any gene known to those skilled in the art, the activity or the presence of which in biological extracts can be easily measured, may generally be used as a reporter gene for implementing the invention. A particularly preferred reporter gene is the β-lactamase gene. The reporter genes are placed under the control of promoters comprising one or more regions imparting sensitivity to one or more signals produced by the activation or the nonactivation of a PAR receptor (calcium, IP3, calmodulin, etc.). These promoters can, moreover, comprise regulatory regions such as enhancers, transcription factor-binding sites, etc.

A particularly preferred reporter system used in the context of the invention comprises a beta-lactamase gene placed under the control of a promoter comprising an NFAT domain sensitive to Ca⁺⁺ ions.

The invention also encompasses a method of producing a cell expressing a modified PAR receptor according to the invention, characterized in that it comprises at least one step of introduction, into a suitable host cell, of a polynucleotide or vector encoding a PAR polypeptide according to the invention, or a fragment of the latter, and optionally the selection of the cells expressing said polypeptide.

The cells can be cultured in any suitable medium and device, for example dishes, bottles, bags, flasks, tubes, etc. The media may be any medium compatible with cell culture, such as commercial media (RPMI, HAM, etc.). The cells can be used to produce the modified PAR polypeptides according to the invention, to test their activity and to identify modulators of the PAR activity.

Another subject of the invention concerns transgenic (nonhuman) animals expressing a modified PAR polypeptide according to the invention. Preferred animals in the context of the invention are nonhuman mammals, such as rodents. Such host animals may comprise a polynucleotide or a vector encoding a modified PAR polypeptide according to the invention, recombinant host cells according to the invention, or else a PAR gene encoding a modified PAR polypeptide, introduced into their genome by homologous recombination.

The production of such transgenic animals is carried out using techniques well known to those skilled in the art (see, for example, U.S. Pat. Nos. 4,873,191, 5,464,764 and 5,789,215 regarding the production of transgenic mice). Generally, the method involves transfecting, into embryonic cells or stem cells, a polynucleotide or expression vector according to the invention, selecting the cells having integrated said polynucleotide or expression vector, and then injecting them or internalizing them into blastocytes subsequently introduced into a female, to give birth to a transgenic animal.

### Production of a Modified PAR Polypeptide

The present invention also relates to methods of preparing a modified PAR polypeptide according to the invention. These methods essentially comprise the production of polypeptides by the recombinant pathway, enzymatic digestion, chemical synthesis, or a combination of these methods. The modified PAR polypeptides according to the invention can be produced in isolated or purified form.

The present invention thus relates to a method of preparing a modified PAR polypeptide according to the invention, characterized in that it comprises at least:

a) obtaining a polynucleotide encoding said modified PAR polypeptide;

b) inserting said polynucleotide into an expression vector, said polynucleotide being functionally linked to a promoter;

c) producing said modified PAR polypeptide from said polynucleotide; and

d) optionally isolating and/or purifying said modified PAR polypeptide thus produced.

In order to ensure, during step c), effective expression of the polynucleotide encoding a modified PAR polypeptide according to the invention, said polynucleotide may comprise signals which promote the transfer of the PAR receptor to the cell membrane, to be inserted therein in a functional form. Such signals are naturally present in the sequence of the PAR gene but can be replaced and/or supplemented with heterologous signals originating from other membrane-bound proteins, such as the signal peptide present in proopiomelanocortin (POMC) for example.

The production of said modified PAR polypeptide may be carried out in vitro or in a cell system. In vitro transcription and translation systems are well known to those skilled in the art. The transcription of said polynucleotide can, for example, be carried out in vitro in a suitable buffer using bacterial RNA polymerases such as SP6, T3 or T7 if said polynucleotide has been inserted into an expression vector under the control of SP6, T3 or T7 promoters respectively (Sambrook and Russell, (2001) above, 9:87-9:88). The translation can also be carried out in vitro using translation systems known to those skilled in the art, such as, for example, wheatgerm lysate or rabbit reticulocyte lysate.

The expression of said polypeptide can also be carried out in a cell system after introduction of a suitable expression vector into a host cell by any means known to those skilled in the art, and in particular by transfection, electroporation, microinjection, infection using a viral vector or a bacteriophage containing the sequence(s) of interest, by cell fusion or gene transfer mediated by a chromosome or a microcell, by spheroplast fusion, etc.

The isolating or purifying step d) can be carried out using the in vitro expression system, the host cell or a membrane preparation of said host cell (e.g. using a lysate of the host cell) . The polypeptide thus produced can be isolated, purified or characterized by any known technique, for example by lysis (e.g. chemical, physical, mechanical, etc.), followed by treatment by centrifugation, binding to an affinity chromatography column on which monoclonal or polyclonal antibodies against PAR polypeptides or their fragments have previously been immobilized, etc.

The purification of the modified PAR polypeptides according to the invention, or of their fragments, can also be carried out by passage over a nickel or copper affinity chromatography column. This is the case, for example, of PAR-nickel fusion proteins. According to another embodiment of the invention, the PAR polypeptides or,, peptide fragments thus obtained are purified by reverse-phase HPLC chromatography and/or by cationic exchange. In another particular embodiment of the methods of preparing and producing a modified PAR, the sequence of the receptor comprises a “tag” region which facilitates its detection or its purification.

### Screening

Another subject of the present invention is the use of PAR polypeptides according to the invention (including fragments of the latter) in the context of screening for active compounds. The screening methods can be carried out in vitro or in vivo, on cell systems, animals, membrane preparations, purified polypeptides, etc. The modified PAR polypeptides according to the invention, or the fragments of the latter, can be used in a purified form, like a chimeric protein (such as those described in a phage display), or else in the form of a preparation based on cell membrane(s), intact cells or transgenic (nonhuman) animals.

The present invention also relates, in general, to the use of a polypeptide or of a fragment as described above or of a polynucleotide according to the invention, for selecting, in vitro, ex vivo or in vivo, compounds which modulate the activity of at least one PAR receptor.

Various types of screening method can be applied. It may be a screening method involving a binding assay or a functional screening method.

### Screening Method Involving a Binding Assay

According to a first method variant, the invention relates to a method of screening for, selecting or identifying active compounds, characterized in that it comprises at least:

a) bringing at least one test compound into contact with a modified PAR polypeptide according to the invention, and

b) selecting the test compound(s) which bind(s) to said modified PAR polypeptide.

In this embodiment, the compound is selected on the basis of its ability to bind to a modified PAR polypeptide according to the invention, in particular to the active site of said modified PAR polypeptide. This binding can be detected or measured by various techniques known to those skilled in the art, such as measurement of fluorescence, of optical density (O.D.), of luminescence, etc. This detection may be carried out according to the FRET (Fluorescence Resonance Energy Transfer; see WO 00/37077) technique, the scintillation proximity assay (SPA) technique, using biochips, by immunological methods, affinity chromatography, etc.

In a typical embodiment, the bringing into contact is carried out in the presence of a reference ligand of the wild type PAR receptor, and the ability of the test compound to bind the receptor of the invention is detected or measured by the displacement of the binding of the reference ligand. The reference ligand may be an exogenous synthetic peptide, an antibody, etc.

In a particular embodiment, the test compound (or the reference ligand) may be the subject of labelling, allowing its binding (or its displacement) to be measured. This may be fluorescent labelling using fluorescein, Oregon Green, Rhodamine, Texas Red, Bodipy or Cyanine or its derivatives. A fluorimeter then makes it possible to detect the labelled compounds or ligands bound to the receptor. Polarization of the fluorescence can also make it possible to distinguish the bound compounds (or ligands) from the free compounds (or ligands) without having to separate them, since the former are less mobile than the latter. The test compound (or the reference ligand) can also be labelled with iodine 125 by addition of a pre-iodinated reactive group or by direct iodination of the compound (or ligand) on a tyrosine residue, for example. The labelling can also be carried out using tritium, using the Bolton and Hunter reagent, or by attachment of an organic radical which is small in size, etc.

For this first method variant, it is possible to use an isolated or a purified, modified PAR polypeptide, in free form or optionally immobilized on a support (column, bead, dish, liposome, etc.). It is also possible to use an intact cell expressing such a modified PAR polypeptide, or a preparation or a membrane extract of such a cell. Preferably, the modified PAR polypeptide(s) is (are) expressed by cells or included in cell extracts or membrane fragments, or a mixture thereof.

According to a particularly preferred embodiment of the invention, the test compound is brought into contact with a modified PAR polypeptide (or a cell or a membrane preparation expressing a PAR polypeptide) comprising a sequence chosen from the group consisting of sequence SEQ ID No:4 (PAR-2Mut2), sequence SEQ ID NO:5 (PAR-2Mut3) and sequence SEQ ID NO:6 (PAR-2Mut4). It is also possible to use functional variants or fragments of such modified PAR polypeptides.

In one embodiment of this first method variant, the present invention provides a method of screening for, selecting or identifying active compounds, characterized in that it comprises the steps of

(a) bringing into contact a modified PAR polypeptide of the invention, and a test compound ; and

(b) adding a radiolabelled ligand of reference to the mixture obtained in step (a);

(c) measuring the radioactivity; and

(d) comparing the radioactivity obtained at step c) with the radioactivity obtained in the absence of test compound.

In this embodiment, when the modified PAR polypeptide is a PAR-2 polypeptide, the reference ligand is preferably SLIGRL, SLIGKV, SLIGR, propionyl-tc, trans-cinnamoyl-LIGRLO or SFLLR,and is more preferably propionyl-tc. Most preferrably the ligand of reference is [3H]propionyl-tc-NH2.

In a particular embodiment, the modified PAR polypeptide is included in membrane fragments.

In a second embodiment of this first method variant, the present invention encompasses a method of screening for, selecting or identifying active compounds, characterized in that it comprises the steps of

(a) providing a desired amount of a modified PAR polypeptide of the invention;

(b) adding the desired amount of a polypeptide provided in step a) to a buffer solution containing a desired amount of a test compound;,

(c) adding a radiolabelled ligand of reference to the mixture obtained in step (b); and

(d) comparing the radioactivity obtained at step c) with the radioactivity obtained in absence of test compound.

In this embodiment, when the modified PAR polypeptide is a PAR-2 polypeptide, the reference ligand is preferably SLIGRL, SLIGKV, SLIGR, propionyl-tc, trans-cinnamoyl-LIGRLO or SFLLR,and is more preferably propionyl-tc. Most preferrably the ligand of reference is [3H]propionyl-tc-NH2.

In a particular embodiment, the modified PAR polypeptide is included in membrane fragments.

In a third embodiment of this first method variant, the present invention encompasses a method of screening for, selecting or identifying active compounds, characterized in that it comprises the steps of:

(a) providing a radiolabelled ligand of reference;

(b) adding the desired amount of a ligand of reference provided in step a) to a buffer solution containing a desired amount of a test compound;

(c) adding a modified PAR polypeptide of the invention to the mixture obtained in step (b); and

(d) comparing the radioactivity obtained at step c) with the radioactivity obtained in the absence of test compound.

In this embodiment of this first method variant, when the modified PAR polypeptide is a PAR-2 polypeptide, the reference ligand is preferably SLIGRL, SLIGKV, SLIGR, propionyl-tc, trans-cinnamoyl-LIGRLO or SFLLR,and is more preferably propionyl-tc. Most preferrably the ligand of reference is [3H]propionyl-tc-NH2.

In a particular embodiment, the modified PAR polypeptide is included in membrane fragments.

In a further embodiment, the present invention provides a method of screening for, selecting or identifying active compounds, characterized in that it comprises the steps of

a) bringing into contact a radiolabelled ligand of reference, a test compound and a modified PAR polypeptide of the invention;

b) bringing into contact into reference test tubes or reference wells of the microplates, a radiolabelled ligand of reference, a high concentration of PAR ligand of reference (for non specific binding) or no compound (for total signal), and a modified PAR polypeptide of the invention;

c) Separating the bound radioligand from the free radioligand by rapid filtration;

d) Adding a scintillation cocktail on the filters and measuring the radioactivity;

e) Comparing the specific signal (Total signal non specific signal) obtained without any test compound to the specific signal obtained with the compound (signal in conditions a)—non specific signal).

In this embodiment of this first method variant, when the modified PAR polypeptide is a PAR-2 polypeptide, the reference ligand is preferably SLIGRL, SLIGKV, SLIGR, propionyl-tc, trans-cinnamoyl-LIGRLO or SFLLR,and is more preferably propionyl-tc. Most preferrably the ligand of reference is [3H]propionyl-tc-NH2.

In a particular embodiment, the modified PAR polypeptide is included in membrane fragments.

### Functional Screening Method

According to a second method variant, the invention relates to a method of functional screening in which the activity of a PAR receptor is measured subsequent to the binding of a test compound (alone or optionally in competition with a ligand). The invention therefore also relates to a method of screening for, selecting or identifying active compounds, characterized in that it comprises at least:

a) bringing a test compound into contact with a modified PAR polypeptide according to the invention, and

b) detecting the activity of said modified PAR polypeptide.

In this embodiment, the modified PAR polypeptide is typically expressed by a cell (in culture) or by a transgenic animal. The bringing into contact is therefore advantageously carried out in vitro or ex vivo, by incubation of the cells with the test compound, or in vivo, by administration of the test compound to a transgenic animal.

According to a particularly preferred embodiment, the test compound is brought into contact with a cell, a membrane preparation or a transgenic animal expressing a modified PAR polypeptide, preferentially expressing a modified PAR polypeptide encoded by a sequence chosen from the group consisting of sequence SEQ ID NO:4 (PAR-2Mut2), sequence SEQ ID NO:5 (PAR-2Mut3), sequence SEQ ID NO:6 (PAR-2Mut4), and their functional variants and fragments.

In one embodiment of this second method variant, the present invention provides a method of screening for, selecting or identifying active compounds, characterized in that it comprises the steps of

a) providing a recombinant host cell expressing a modified PAR polypeptide of the invention, in an appropriate culture medium;

b) adding a desired concentration of a test compound in said culture medium;

c) measuring the activity of said modified PAR polypeptide.

In another embodiment, the present invention encompasses a method of screening for, selecting or identifying active compounds, characterized in that it comprises the steps of

a) providing a recombinant host cell expressing a modified PAR polypeptide of the invention, in an appropriate culture medium;

b) adding a desired concentration of a test compound in said culture medium;

c) adding a reference ligand in said culture medium obtained at step b); and

d) measuring the activity of said modified PAR polypeptide; and

e) comparing the activity of said modified PAR polypeptide obtained at step d) with the activity of said modified PAR polypeptide obtained when step b) is omitted.

Generally, the host cell cultivated at steps a) of the functional screening assays above may be any culturable host cell as mentioned above, and preferably a host cell of mammalian origin. In a first preferred embodiment of the screening methods above, the recombinant host cell consists of a CHO cell line.

A most preferred recombinant host cell, used in the functional screening assays above, expresses a modified PAR polypeptide encoded by a sequence chosen from the group consisting of sequence SEQ ID NO:4 (PAR-2Mut2), sequence SEQ ID NO:5 (PAR-2Mut3), sequence SEQ ID NO:6 (PAR-2Mut4), and their functional variants and fragments.

When the modified PAR polypeptide is a PAR-2 polypeptide, the reference ligand is preferably SLIGRL, SLIGKV, SLIGR, propionyl-tc, trans-cinnamoyl-LIGRLO or SFLLR,and is most preferably SLIGRL.

The activity of the modified PAR receptor can be revealed by demonstrating the activity of one or more G proteins bound to said receptor, or else of secondary effectors of said G proteins, such as cAMP, IP₃, calcium, diacylglycerols (DAGs), etc., in turn acting on enzymes, ion channels, transporters, etc. These various secondary effectors can themselves be the subject of a labelling similar to those described above. Advantageously, the cell used in the method, expressing the modified PAR receptor, also comprises a reporter system as defined above, for determining the activity of the receptor.

In the context of the invention the secondary effector is preferably calcium. In a most preferred embodiment of the invention, the screening assay is a fluorescence assay.

### Reporter Gene Assay

In one embodiment of the invention, the functional screening method of the invention is a reporter gene assay, which permits indirect PAR polypeptide activity measurement.

In another embodiment, the present invention concerns a method of screening for, selecting or identifying active compounds, characterized in that it comprises the steps of:

a) providing a recombinant host cell coexpressing a modified PAR polypeptide of the invention in an appropriate culture medium and a reporter gene as defined above;

b) adding a desired concentration of a test compound in said culture medium;

c) measuring the reporter gene expression.

In a further embodiment, the present invention also embodies a method of screening for, selecting or identifying active compounds, characterized in that it comprises the steps of:

a) providing a recombinant host cell coexpressing a modified PAR polypeptide of the invention in an appropriate culture medium and a reporter gene as defined above;

b) adding a desired concentration of a test compound in said culture medium;

c) adding a reference ligand in said culture medium obtained at step b);

d) measuring the reporter gene expression; and

e) comparing the reporter gene expression obtained at step d) with the reporter gene expression when step b) is omitted.

Generally, the host cell cultivated at steps a)above may be any culturable host cell as mentioned above, and preferably a host cell of mammalian origin. In a first preferred embodiment of the screening methods above, the recombinant host cell consists of a CHO cell line.

A most preferred recombinant host cell, used in the assay above, expresses a modified PAR polypeptide encoded by a sequence chosen from the group consisting of sequence SEQ ID NO:4 (PAR-2Mut2), sequence SEQ ID NO:5 (PAR-2Mut3), sequence SEQ ID NO:6 (PAR-2Mut4), and their functional variants and fragments.

When the modified PAR polypeptide is a PAR-2 polypeptide, the reference ligand is preferably SLIGRL, SLIGKV, SLIGR, propionyl-tc, trans-cinnamoyl-LIGRLO or SFLLR,and is most preferably SLIGRL.

In a preferred embodiment, the reporter gene assay permits Ca++ measurement.

In preferred embodiments of the invention the screening assay uses a reporter gene as described above.

In a preferred embodiment of the invention the reporter gene is the β-lactamase gene.

In a most preferred embodiment the reporter gene is the β-lactamase gene placed under the control of a promoter comprising an NFAT domain sensitive to Ca++ ions.(see for example Zlokarnik et al., 1998, Science, 279(5347) :84-8.)

### Principles of the Fluorescence Energy Transfer (FRET) Gene Reporter-Based Assay

A cell-based assay suitable for HTS has been set up using the stably transfected CHO cell line and the beta-lactamase reporter gene technology from Aurora Biosciences. This technology is based on the use of a gene reporter to detect variations of a second messenger intracellular concentration. In the present case, Icrac channel stimulation leads to an oscillatory intracellular Ca²⁺ influx, which triggers the activation of the NFAT promoter leading to the synthesis of beta-lactamase molecules. The enzyme activity is monitored using a fluorescent substrate containing a beta-lactamase ring (CCF2-AM, emitting at 535 nm), which, once cleaved, emit a blue light (460 nm) . The ratio of the fluorescence unit (F.U.) measured at 460 over the F.U. at 535 nm is dependent upon the number of beta-lactamase molecules, but not upon the number of cells.

### Direct PAR Polypeptide Activity Measurement Assay

In another embodiment of the invention the functional screening method of the invention is an assay which permits direct PAR polypeptide activity measurement.

In a preferred embodiment, the direct PAR polypeptide activity assay permits Ca++ measurement.

In a most preferred embodiment of the invention the functional screening method uses the FLIPR technology. The Fluorometric Imaging Plate Reader (FLIPR) (Molecular Devices Corporation, Sunnyvale, Calif.) was developped as a screening tool for cell-based fluorescent assays (Sullivan et al., Methods in molecular biology, vol 114: calcium signaling protocols, David Lambert, ed. Humana Press (1999), pages 125-133). It allows the simultaneous stimulation and, measurement of 96 separate cell samples. Therefore, it is possible to quantify transient signals, such as the release of intracellular calcium, in parallel and in real time.

The FLIPR technology features an argon ion laser that provides discrete spectral lines spaced from approximately 350 to 530 nm. For use with fluorescent Ca++ dyes such as fluo-3, fluo-4 and Calcium Green-1, the 488 nm line of the laser is employed. These two dyes exhibit an increase in fluorescence emission intensity on binding to Ca++ and hence are used to detect increases in the level of intracellular calcium.

In a preferred embodiment of the invention the readings were taken at 550 nm. The FLIPR quantifies the release of intracellular calcium in real time, the fluorescence is measured during 90 seconds.

In a first preferred embodiment of the above direct PAR polypeptide activity measurement screening method, the recombinant host cell consists of a CHO cell line.

A most preferred recombinant host cell, used in the assay above, expresses a modified PAR polypeptide encoded by a sequence chosen from the group consisting of sequence SEQ ID NO:4 (PAR-2Mut2), sequence SEQ ID NO:5 (PAR-2Mut3), sequence SEQ ID NO:6 (PAR-2Mut4), and their functional variants and fragments.

When the modified PAR polypeptide is a PAR-2 polypeptide, the reference ligand is preferably SLIGRL, SLIGKV, SLIGR, propionyl-tc, trans-cinnamoyl-LIGRLO or SFLLR,and is most preferably SLIGRL.

In another embodiment of the invention the direct intracellular calcium measurement is performed using systems equivalent to the FLIPR (e.g. FDSS functional drug screening system from Hamamatsu or more classically a spectroflorimeter)

A functional screening assay which permits direct calcium release measurement is preferred over a reporter gene assay.

Whatever the method variant pursued, the test compounds can be brought into contact with the modified PAR receptor(s) (including the cells or animals expressing them) for varying periods of time, depending on their effect(s), their concentration, the nature of the cells, etc.

The contact can be brought about on any suitable support, and in particular on a plate, in a tube or in a bottle. Generally, for in vitro or ex vivo assays, the bringing into contact is carried out in a multiwell plate which makes it possible to perform, in parallel, many and varied assays. Among typical supports are microtitration plates and more particularly 48-well, 96-well or 384-well (or more) plates. Depending on the support and the nature of the test compound, varying amounts of cells can be used in carrying out the methods described. Conventionally, 10³ to 10⁶ cells are brought into contact with a type of test compound, in a suitable culture medium, and preferentially between 10⁴ and 10⁵ cells.

In the methods of the invention, it is possible to test several test compounds with a polypeptide, in parallel, a test compound on several polypeptides, or several test compounds on several polypeptides.

In general, the screening methods of the invention are generally carried out in parallel in the absence of test compound and/or with a reference compound, and the results obtained are compared, making it possible to evaluate the effect of the test compound.

Moreover, the test compounds selected can then be validated in secondary assays, for example with wild-type PAR receptors and/or in animals.

The various described above screening methods of the invention can be run independently. But a described above screening method of the invention can also be used as a secondary assay.

Thus, a test compound which has been positively screened according to one described above screening method of the invention (functional screening method or screening method involving a binding assay (binding method)) may then be tested according to another described above screening method of the invention.

Therefore, the invention also deals with a method of screening for, selecting or identifying active compounds, characterized in that it comprises the steps of:

a) performing a binding method of the invention with a test compound; and if said test compound is found to modulate the PAR polypeptide activity, then

b) performing a functional screening method according to the invention with the modulator compound selected at step a).

The invention also encompasses a method of screening for, selecting or identifying active compounds, characterized in that it comprises the steps of:

a) performing a functional screening method according to the invention with a test compound; and if said test compound is found to modulate the PAR polypeptide activity, then

b) performing a binding method of the invention with the modulator compound selected at step a).

According to the invention, it may also be ensured that the PAR polypeptide activity modulator compounds selected according to any one of the screening, selection or identification methods above is selective for a PAR subtype polypeptide and does not modulate neither other PARs, nor other proteins.

Thus, the invention also pertains to a method for selecting a compound that selectively modulates said PAR polypeptide activity, wherein said method comprises the steps of:

a) selecting a compound which modulates PAR polypeptide activity by carrying out a screening method of the invention; and

b) assaying the selected modulator compound for its inability to inhibit the PAR polypeptide activity of at least one other PAR.

With the screening methods of the invention we can screen for, select or identify agonist or antagonist of PAR receptor.

The invention also relates to a method of determining whether a compound is a PAR agonist, wherein said method comprises the steps of:

a) performing a screening method according to the invention with a test compound; and if said test compound is found to modulate the PAR polypeptide activity, then

b) performing a selection method of the invention, wherein said method comprises the steps of:

c) providing a desired amount of a modified PAR polypeptide of the invention;

d) adding said modified PAR polypeptide to a solution containing a desired amount of said test compound; and

e) determining whether there is a Ca2+ release to confirm whether the test compound that was positively screened according to a screening method of the invention is an agonist of a PAR receptor.

In fact, if there is a calcium release the test compound that was positively screened according to a screening method of the invention is an agonist of a PAR receptor.

A further object of the invention is a PAR polypeptide modulator compound which has been selected according to any one of the methods described above.

The methods according to the invention preferably make it possible to select one or more active compounds which modulate the activity of at least one PAR receptor. The expression “compound which modulates” should be understood to mean any compound capable of activating or inhibiting a PAR receptor, in particular any agonist or antagonist of a PAR receptor. These are preferentially selective modulators, i.e. modulators having no significant direct action on another particular membrane receptor. The test compounds can be peptides, nucleic acids, organic or inorganic chemical molecules, lipids, saccharides, collections of chemical compounds.

The present invention also relates to the methods of preparing pharmaceutical compositions involving the use of one or more compounds which modulate the activity of at least one PAR receptor selected according to one of the methods described above.

The invention relates in particular to a method of preparing a pharmaceutical composition, comprising (i) selecting an active compound according to one of the methods described above, and (ii) conditioning this compound, or an analogue thereof, with a pharmaceutically acceptable excipient or vehicle.

### Uses of the PAR Polypeptides and/or of Their Ligans(s)

Another subject of the invention concerns the use of a polypeptide or of a nucleic acid molecule according to the invention, for preparing a composition intended to modulate or restore the activity of PAR receptors in vivo.

It also concerns the use of a compound identified using a method according to the invention, for producing a composition intended to modulate the activity of PAR receptors in vivo, in particular for the treatment of inflammation, of allergies, of diseases affecting the CNS, of neurodegeneration, or of psychiatric or cardiovascular diseases.

Other aspects and advantages of the present invention will become apparent on reading the examples which follow, which should be considered to be illustrative and nonlimiting.

### LEGEND OF THE FIGURES

FIG. 1. Mechanism of activation of a PAR by enzymatic cleavage. The organization of the 7 transmembrane domains of the PAR proteins in the plasma membrane is shown, as is their association with the G proteins (oval). The endogenous ligand released by proteolytic cleavage is represented by a black box. The terms EC, IC and TM signify, respectively, extracellular, intracellular and transmembrane.

FIG. 2. Examples of truncated PAR-2 receptors.

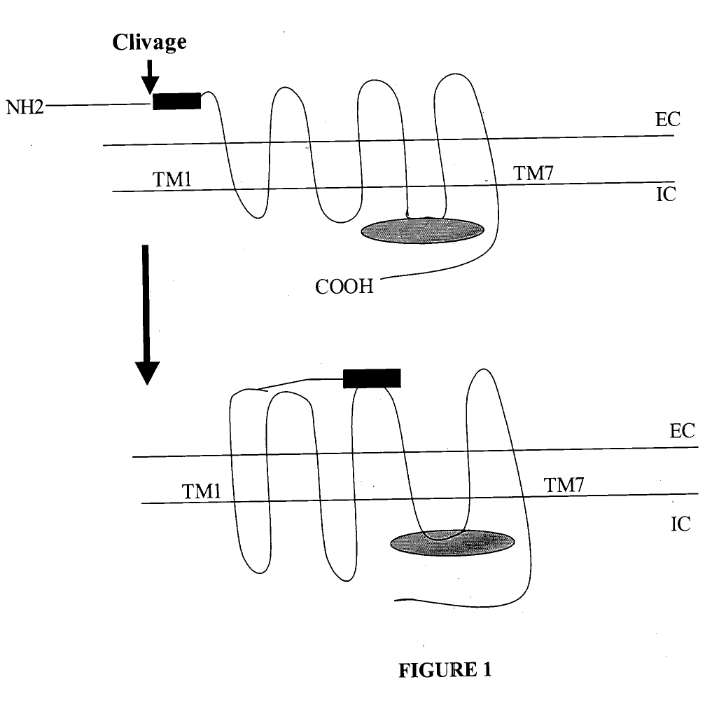

The proteolytic cleavage site (CS) is indicated, as are the first two transmembrane domains (TMI and TMII) . The sense Bam HI and antisense Sfi primers which can be used to construct the vectors Mut2, Mut3 and Mut4 are indicated. The Mut2, Mut3 and Mut4 constructs are modified PAR-2 polypeptides according to the invention.

Mut2: deletion of the endogenous activating ligand. Mut 2 corresponds to a deletion from amino acid 1 to 12 of the mature wild type PAR-2.

Mut3: deletion of the first 20 amino acids of the N-terminal region, this being calculated from the first aminoacid of the vector construction. Mut 3 corresponds to a deletion from amino acid 1 to 26 of the mature wild type PAR-2.

Mut4: deletion of the first 40 amino acids of the N-terminal region preceding TM1, this being calculated from the first aminoacid of the vector construction.Mut 4 corresponds to a deletion from amino acid 1 to 37 of the mature wild type PAR-2.

In the present application, if not stated otherwise, all the numbering of amino acids, and amino acid ranges are calculated from the first amino acid of the PAR precursor.

FIG. 3: Strategy adopted for constructing the vector PAR-2Mut2.

The initial vector POMC-Flag-PAR-2-12CA5 encodes the signal peptide of proopiomelanocortin, or POMC (in black), contains a Flag epitope (in white), part of the pre-pro region of the PAR-2 precursor (squares), and also the mature PAR-2 protein (endogenous ligand (dots), amino acids 43 to 120 (wavy lines) and 121 to 397 (hatched)). The site of cleavage by a protease is indicated by “CS”. The natural Sfi I restriction site is indicated, as is the Bam HI restriction site created by mutagenesis. The sense (BAM-HI S) and antisense (Sfi I AS) PCR primers used to amplify the region encoding amino acids 43 to 120 of PAR-2 and comprising Bam HI, in the 5′ position, and Sfi I, in the 3′ position, restriction sites (Mut 2 PCR fragment) are indicated.

FIG. 4: Activity of modified PAR receptors.

FIG. 4A: Outline of points representative of the labelling produced on the CHO-PAR-2Mut2 cells. The unstimulated CHO-PAR-2Mut2 cells appear on the left-hand table and the stimulated cells (stimulated with 30 μm of a synthetic ligand, SLIGRL-NH₂) appear on the right-hand table.

FIG. 4B: Outline of points representative of the labelling produced on the CHO-PAR-2Mut2 cells.

For FIGS. 4A and 4B, the cells emitting a beta-lactamase signal are isolated, cultured and recovered by virtue of selection involving a nonconstitutive beta-lactamase for the purpose of obtaining monoclones (region R3 of the right-hand table).

FIG. 5: Fluorescent emission in the CHO-NFAT-PAR-2WT and CHO-NFAT-PAR-2Mut2 cells after stimulation with an exogenous peptide SLIGRL-NH₂: concentration-effect curve. Each point represents the mean ratio of the fluorescent response (±SEM, bars) for 5 samples of replicated cells derived from the same monoclone.

FIG. 6: Fluorescent emission in the CHO-NFAT-PAR-2WT and CHO-NFAT-PAR-2Mut2 cells after stimulation with trypsin: concentration-effect curve. Each point represents the mean ratio of the fluorescent response (±SEM, bars) for 5 samples of replicated cells derived from the same monoclone.

### EXAMPLES OF IMPLEMENTATION

### A) Construction of Truncated PAR-2 Receptors

### A1) Construction of a Truncated PAR-2 Receptor (PAR-2 Mut2)

### a) Construction of a Vector PAR2Bam

The expression vector POMC-tag-PAR-2-12CA15 (FIG. 3) is a pcDNA3 vector containing a cDNA encoding amino acids 31 to 397 of the precursor of the human PAR-2 protein and also comprising an N-terminal tag epitope (Bohm et al. (1996b) J. Biol. Chem. 271:22003-22016) and also the signal sequence of proopiomelanocortin (amino acid 1 to 26), in order to ensure correct insertion into the membrane of the host cell. This vector was modified as follows:

A BamHI restriction site was inserted by site-directed mutagenesis into POMC-tag-PAR-2-12CA15, thus changing an adenine for a guanine in the sequence encoding POMC-tag-PAR-2 (see FIG. 3). This change also involved an amino acid change of an arginine to a glycine (at position 31 of the sequence of the PAR-2 precursor) . The mutagenesis was performed using the Stratagene kit (quick change site directed mutagenesis kit, Cat #200519) and the sequence primers (Eurogentec) SEQ ID NOs: 7 and 8, following the manufacturer's instructions. After transfection, into E. coli DH5α competent cells, of the mutagenesis product (Life Technologies Cat #18258012), screening by sequencing was carried out (applied biosystems 373 sequencer) in order to identify the clones expressing the expected Bam HI mutation. The plasmid thus mutated was named PAR-2Bam.

PAR-2Bam was then digested with the BamHI and SfiI restriction enzymes (see FIG. 3). Two fragments, respectively 6400 bp and 260 bp, were thus generated. The 6400 bp fragment, which was then used to construct the vector PAR-2Mut2, was purified using the Quiaquick kit (Quiagen Cat #28704) after separation by electrophoresis on a gel containing 1% agarose and visualization under UV light using ethidium bromide.

### b) Construction of the Vector PAR-2Mut2

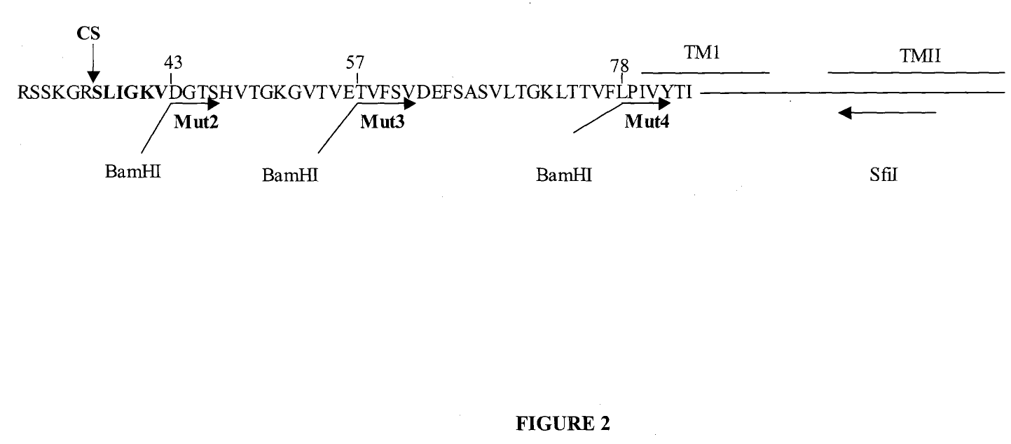

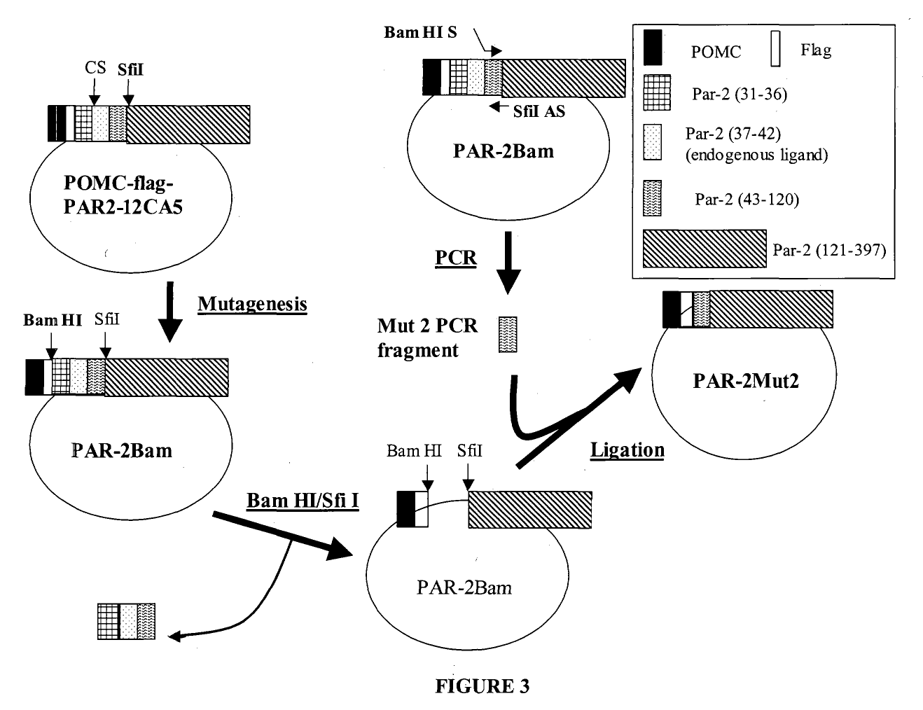

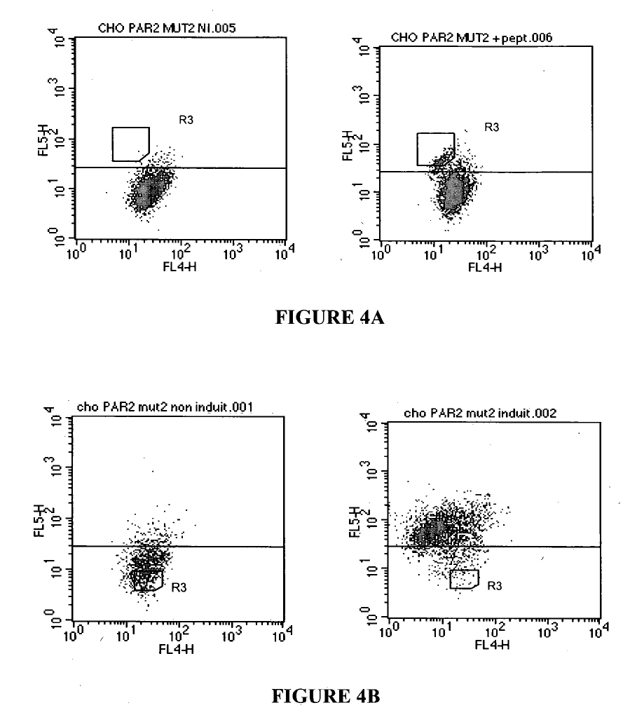

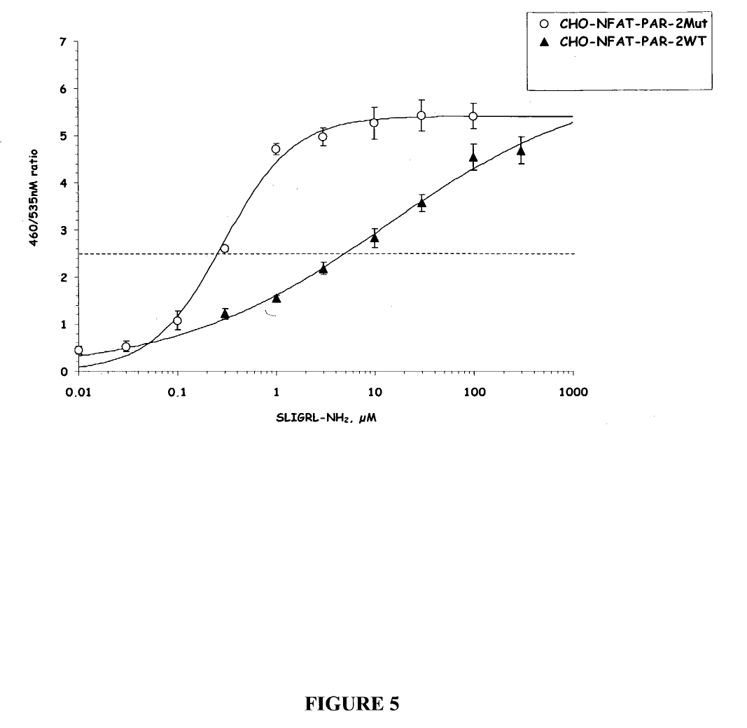

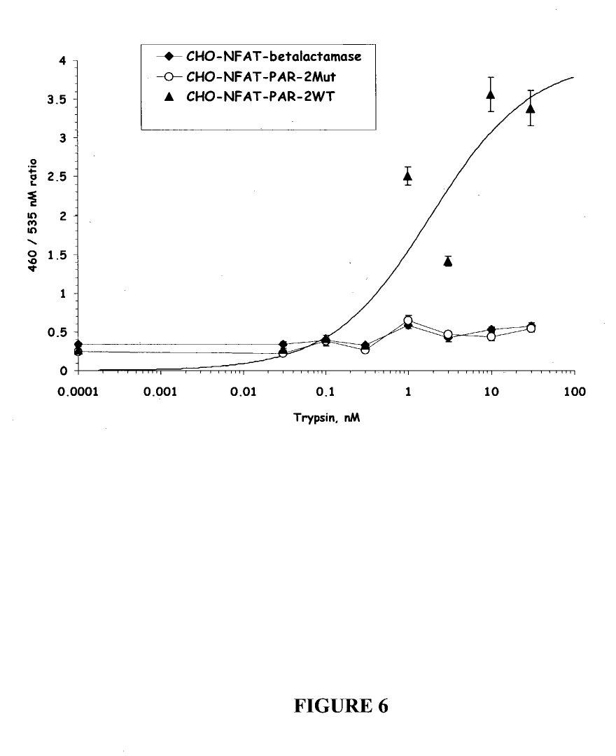

The truncated receptor PAR-2Mut2 was then obtained in the following way. A PCR amplification of the plasmid PAR-2Bam with the 2 primers sense Bam HI of SEQ ID NO: 9 and antisense Sfi of SEQ ID NO:10 was carried out in order to amplify a 230 bp fragment encoding amino acids 43 to 121 of the precursor of human PAR-2 (see FIGS. 2 and 3). The primer of SEQ ID NO:9 also contains an additional BamHI restriction site in its 5′ portion. The primer of SEQ ID NO:10 hybridizes to a region of PAR-2 containing the SfiI site. The PCR conditions were as follows: 94° C. for 30 seconds, 56° C. for 30. seconds, 72° C. for 1 minute, for 30 cycles carried out in the presence of a “Hi-Fidelity” Taq Polymerase (Roche Cat #1732641).

The 230 bp BamHI-SfiI fragment thus amplified was cloned into a plasmid using the PCR-Blunt kit (Invitrogen Cat #K270020) . A positive clone containing the 230 bp insert was digested with BamHI and SfiI and then ligated using T4 DNA ligase (Roche Cat #0799009), and maintained overnight at 14° C., into the vector PAR-2Bam digested with the same set of enzymes and purified as described above.

DH5α cells were then transformed with the ligation.

A PAR-2Mut2 clone was then sequenced. Its sequence corresponded directly to the expected truncated receptor, no longer comprising an endogenous ligand peptide, but indeed comprising amino acids 43 to 397 of the precursor of human PAR-2 (see FIGS. 2 and 3 and SEQ ID NO:4).

### A2) Construction of a Truncated PAR-2 Receptor (PAR-2 Mut3)

The truncated receptor PAR-2Mut3 was then obtained in the following way. A PCR amplification of the plasmid PAR-2Bam with the 2 primers sense Bam HI of SEQ ID NO:11 and antisense Sfi of SEQ ID NO:10 was carried out in order to amplify a 203 bp fragment encoding amino acids 57 to 121 of the precursor of human PAR-2. The primer of SEQ ID NO:11 also contains an additional BamHI restriction site in its 5′ portion. The primer of SEQ ID NO:10 hybridizes to a region of PAR-2 containing the SfiI site. The PCR conditions were as follows: 94° C. for 30 seconds, 56° C. for 30 seconds, 72° C. for 1 minute, for 30 cycles carried out in the presence of a “Hi-Fidelity” Taq Polymerase (Roche Cat #1732641).

The 187 bp BamHI-SfiI fragment thus amplified was cloned into a plasmid using the PCR-Blunt kit (Invitrogen Cat # K270020) . A positive clone containing the 187 bp insert was digested with BamHI and SfiI and then ligated using T4 DNA ligase (Roche Cat #0799009), and maintained overnight at 14° C., into the vector PAR-2Bam digested with the same set of enzymes and purified as described above.

DH5a cells were then transformed with the ligation.

A PAR-2Mut3 clone was then sequenced. Its sequence corresponded directly to the expected truncated receptor, no longer comprising an endogenous ligand peptide, but indeed comprising amino acids 57 to 397 of the precursor of human PAR-2 (see SEQ ID NO:5).

### B) Establishment of Stable Cell Lines

### B1). CHO-NFAT-PAR-2-Mut2

CHO-NFAT-WT cells (CHOs stably transfected with a beta-lactamase gene under the control of NFAT regulatory sequences, Aurora) were cultured in a Glutamax I DMEM medium containing 4.5 g/l of glucose (LifeScience, Gaithersburg, Md., ref. #61965-026) supplemented with 10% of foetal calf serum and gentamycin at 100 μg/ml. 250 μg/ml of zeomycin and 150 μg/ml of hygromycin were added to the medium to allow stable propagation of the cells.

10 μg of the expression vector PAR-2Mut2 were introduced into CHO-NFAT-WT cells using the Fugene technique (Roche, Cat #181443). The stable transfectants were then selected in the presence of geneticin (Life Technologies Cat #11811049) in a proportion of 500 μg/ml and zeomycin in a proportion of 250 μg/ml, 24 hours after transfection.

After selection for three weeks, the cells were sorted by FACS using the CCF4/beta-lactamase reporter system (AURORA Technology) and subsequent to stimulation carried out with the SLIGRL peptide present at 30 μM (see FIG. 4A). Thus, the production of beta-lactamase, subsequent to activation of the PAR2 receptor inducing the production of calcium which activates NFAT, is detected by the CCF4/AM fluorescent probe, producing fluorescent emissions which are different depending on whether it is intact or cleaved by the beta-lactamase (WO 96/30540)

The cells emitting a beta-lactamase signal were then isolated, cultured and sorted by virtue of selection involving a nonconstitutive beta-lactamase for the purpose of obtaining monoclones (see FIG. 4B).

### B2) CHO-NFAT-PAR-2-Mut3

The same protocol as in B1) has been used with the difference that 10 μg of the expression vector PAR-2Mut3 were introduced into CHO-NFAT-WT cells using the Fugene technique (Roche, Cat #181443)

### B3) CHO-NFAT-PAR-2-WT

The plasmid containing the cDNA coding for PAR2WT has been obtained by University of San Francisco. CHO-NFAT-WT, and the same protocol as described in B1) have been used.

### C) Beta-Lactamase Assay

### C1) CHO-NFAT-PAR2Mut2 Cell Line Test

In a 96-well or 384-well microplate, the SLIGRL-NH₂ synthetic ligand (Neosystème, France) was used to stimulate the CHO-NFAT cells expressing the wild-type PAR-2 receptor (CHO-NFAT-PAR-2WT) or the CHO-NFAT cells expressing the PAR-2Mut2 receptor (CHO-NFAT-PAR-2mut2), in a proportion of 4×10⁴ for a 96-well plate or 1×10⁴ for a 384-well plate, for 4 hours, at 37° C., in an atmosphere containing 5% CO₂ and in a modified Dulbecco medium (Lifetech, Gaithersburg, Md.) containing 100 μg/ml of gentamycin. The cells were placed in the dark for 1 hour at ambient temperature with 12 μM of CCF4-AM (Aurora Biosciences, San Diego, Calif.). The fluorescence was measured with a Fluostar fluorimeter (BMG, Germany). The excitation occurs at 405 nm and the emission is recorded at both 460 and 535 nm. The fluorescence ratio (460 nm/535 nm) is estimated after elimination of the background noise (medium without cells). The ED₅₀ is considered to be the concentration of SLIGRL-NH₂ necessary to achieve 50% of the maximum fluorescence ratio.

The CHO-NFAT-beta-lactamase cells stimulated with SLIGRL-NH₂ in the medium mentioned above (4×10⁴ cells/well) were used as control. All the reagents were added with the activating agent (chemical or referenced compounds), unless otherwise indicated.

### Results

The fluorescence obtained in the CHO-NFAT-PAR2WT cells stimulated with increasing concentrations of SLIGRL-NH₂ (from 0.1 μM to 1000 μM) was compared to that obtained in the CHO-NFAT-PAR-2Mut2 cells (see FIG. 5). The ED₅₀ obtained with the CHO-NFAT-PAR2WT cells is 10±5 μM (n=5). Such a value is in agreement with the literature (Al-Ani et al., (1996) above). The dose-effect curve obtained with the CHO-NFAT-PAR-2Mut2 cells was shifted to the left and the ED₅₀ is considerably lower (0.2 μM±0.05; n=5).

Unlike the control CHO-NFAT-PAR2WT cells, the CHO-NFAT-PAR-2-Mut2 cells, like the CHO-NFAT cells, are not activated by trypsin (see FIG. 6), which confirms that such a mutant of the PAR-2 receptor possesses no type of attached ligand which might be activated by proteolytic cleavage.

### C2) Optimized Beta-Lactamase Test

For specifics regarding the handling, storage and preparation of CCF4-AM, as well as technical specificity (filters) for the fluorimeter please refer to the fluorescent bioassay systems technology transfer manual (Fluostar fluorimeter).

Cell culture: CHO-NFAT-PAR2mut2 were seeded in 225 ml flask (40 millions/200 ml) using DMEM complete glutamax medium (ref: 61965-026 Invitrogen) supplemented with 10% foetal calf serum, geneticin (500 μg/ml, ref: 10131-019 Invitrogen) and zeocine (250 μg/ml, ref: 10131-027 Invitrogen). Classically for 50 plates/day pace, 20 flasks were seeded and passed every day. Versene (ref: 15040-033 Invitrogen) was used to pass the cells.

### Experiment

The day before the experiment, the cells were seeded into 384 well plate (COSTAR #3712) in a DMEM complete medium (50 μl) at 25000 cells/well (manually, multichannel pipette). Incubation took place at 37° C. 5% CO2.

The day of the experiment, the medium was discarded (by turning the plate and smooth banging). 45 μl DMEM serum was added to each well. 1.4 μl compound to be tested (250 μM, 25% DMSO) and 20 μl 10.5 μM SLIGRL (Neosystem, ref: SP001568C) were added using the Minitrak VI (Packard) . Plates were incubated at 37° C./90% humidity/5% CO2 for 4 hours. Then, 14 μl of CCF4/AM (Panvera ref: K1030) solution was added to each well (manually, multi channels pipette). Reading (LJL Analyst or BMG Fluostar) was performed as described in C1) after 3 h incubation at room temperature.

### Controls

A blank control (no cells, 1.4 μl DMSO 25%+20 μl DMEM), a MIN control (without SLIGRL, 1.4 μl DMSO 25%+20 μl DMEM) and a MAX control (1.4 μl DMSO 25%+20 μl SLIGRL 10.5 μM) were used.

### Preparation of the 10.5 μM SLIGRL Solution

2 mg of peptide+16.66 ml sterile distilled water+141.43 ml DMEM are mixed.

### Preparation of the CCF4/AM Solution

700 μl of CCF4/AM 1 mM was mixed with 7 ml of solution B (Aurora) and with 108 ml of solution C (Aurora) Compounds which have been positively screened according to this screening assay were then confirmed using the same technique.

To determine whether the confirmed compounds which have been positively screened according to the beta-lactamase screening assays described above, have a specific effect on the PAR2 receptor, 3 assays on FLIPR and an enzymatic assay were developped.

### D) Calcium Release Direct Measurement (FLIPR Assays)

These assays were used as secondary assays to reconfirm the compounds which have been positively screened according to the betalactamase assays described above but they could also be used as primary assays.

The cells used for these tests were: CHO-NFAT-PAR-2-MUT2, CHO-NFAT-PAR-2-MUT3, CHO-NFAT-PAR2-WT and CHO-NFAT-WT.

### D1) PAR2 Activation, using SLIGRL 3 μM/CHO-NFAT-PAR2-MUT2

The day before the experiment, the cells, were seeded into 384 well-plate in a medium DMEM glutamax (ref: 61965-026 Invitrogen, fetal calf serum 10% (ref: 10270106 Invitrogen), gentamycin 50 mg/ml 0.25% (ref: 15750-037 Invitrogen) at 25000 cells/well/50 μL.

The day of the experiment, the medium was discarded (by turning the plate). 50 μl of HBSS (Invitrogen réf: 14025.050), hepes (Invitrogen ref: 15630-05620 mM) were added to each well, HBSS was then discarded by turning the plate. 10 ml of Calcium kit loading solution and 10 ml HBSS, hepes 20 mM and 200 μl probenecid 250 μM were mixed and then 30 μl of this solution was added per well; plates were then incubated during 2 hours at 37° C. 4% CO2. 1.2 μl of a 250 μM compound in 25% DMSO to be tested and 3.8 μl HBSS, hepes 20 mM were added (on the Minitrak Va., Packard), the plates are transferred to the FLIPR and 25 μl 7.2 μM SLIGRL were added.

The dye included in the calcium kit loading solution exhibits an increase in fluorescence emission intensity on binding to Ca++ and hence is used to detect increases in the level of intracellular calcium. The dye is excited with the 488 nm excitation line of the argon laser and emits at 550 nm. The FLIPR quantifies the release of intracellular calcium in real time, the fluorescence is measured during 90 seconds.

### Controls

A MIN control (CHO-NFAT-WT+30 μl calcium loading solution+1.2 μl of HBSS, hepes 20 mM in 25% DMSO+3.8 μl HBSS, hepes 20 mM+25 μl 7.2 μM SLIGRL) and a MAX control (CHO-NFAT-PAR2-Mut2+30 μl calcium loading solution+1.2 μl of HBSS, hepes 20 mM in 25% DMSO+3.8 μl HBSS, hepes 20 mM+25 μl 7.2 μM SLIGRL) were used.

### Preparation of the Calcium Kit Loading Solution (Molecular Devices réf :R8033)

In a bottle of calcium kit, 10 ml HBSS, hepes 20 mM, were added, and the bottle was mixed. 90 ml of HBSS, hepes 20 mM were added and the solution was aliquoted in 10 ml. This solution represented the calcium kit loading solution.

### Preparation of the 7.2 μM SLIGRL Solution (Neosystem ref :SP001568C)

1 mg SLIGRL was mixed with 8.355 ml HBSS, hepes 20 mM. Then, 107.68 ml HBSS, hepes 20 mM were added.

### D2) PAR2 Activation, using Trypsin 30 nM/CHO-PAR2-WT

The day before the experiment, cells were seeded into 384 well-plate in a complete medium at 25000 cells/well/50 μL.

The day after, the medium was discarded (by turning the plate). 50 μl of HBSS, hepes 20 mM were added to each well, HBSS was then discarded by turning the plate. This was done twice.

10 ml of Calcium kit loading solution (solution prepared as described above) and 10 ml HBSS, hepes 20 mM and 200 μl probenecid 250 μM were mixed, and 30 μl of this solution per well were added, plates were incubated during 2 hours at 37° C. 5% CO2.

1.2 μl of compound to be tested (250 μM, 25% DMSO) and 3.8 μl HBSS, hepes 20 mM were added (on the Minitrak Va.). 25 μl 72 nM Trypsin was then added using the FLIPR.

The fluorescence was measured on the FLIPR as specified above.

### Controls

A MIN control (CHO-NFAT-WT+30 μl calcium loading solution+1.2 μl of HBSS, hepes 20 mM in 25% DMSO+3.8 μl HBSS, hepes 20 mM+25 μl 7.2 μM SLIGRL) and a MAX control (CHO-NFAT-PAR2-WT+30 μl calcium loading solution+1.2 μl of HBSS, hepes 20 mM in 25% DMSO+3.8 μl HBSS, hepes 20 mM+25 μl 7.2 μM SLIGRL) were used.

### Preparation of the 72 nM Trypsin Solution

1 mg trypsin (Sigma réf: T8802) and 41.6 ml HBSS, hepes 20 mM were mixed.

7 ml of this solution was mixed to 90 ml HBSS, hepes 20 mM, leading to a 72 nM trypsin solution

### D3) FLIPR/Carbacol 5 μM/JURKAT M1 Test

This assay using muscarinic M1 receptor (carbachol-activated GqPCR) activation with carbachol 5 μM/Jurkat M1 cell line was carried out to check that the compounds which have been positively screened according to the D1) and D2) assays do not block non-specifically Gq-coupled receptors.

Cells were transfered into 500 ml centrifuge tubes and centrifuged 5 min at 2000 rpm. The supernatant was discarded, with a sterile Pasteur pipette.

10 ml of HBSS, hepes 20 mM were added to the tube, mixed, cells were counted with a Malassez cell.

A fluorescent probe was prepared: 50 μg of Fluo 3-Am (Interchim F-1242)+22 μL DMSO+22 μL solution C (aurora)+10 ml HBSS, hepes 20 mM; this solution was added to 20 millions of cells, and incubation took place during 1 hour at 37° C. 5% CO2; from time to time the incubation medium was shaked. Cells were centrifuged 5 min at 2000 rpm. The supernatant was discarded with a sterile Pasteur pipette. 100 ml of HBSS hepes 20 mM were added and the cells were centrifuged at 2000 rpm. The supernatant was discarded with a sterile Pasteur pipette and 6 ml of HBSS, hepes 20 mM were added to the cells, to obtain a suspension containing 3,33 millions cells/ml.

1.2 μL of compound to be tested and 3.8 μL HBSS, hepes 20 mM were put in the wells.

In all the wells 30 μL of cell suspension (at 3.33 Millions cell/ml) were added, the plate was then centrifuged during 5 min at 900 rpm.

Then, the experiment took place on the FLIPR, and 25 μL of 5 μM carbacol were added in all the wells. The fluorescence was measured as described in C1).

### Controls

A MIN control (30 μl Jurkat M1 cell suspension+2 μl 150 μM atropin in 6% total DMSO+3 μl of HBSS, hepes 20 mM in 6% total DMSO+25 μl 5 μM carbacol) and a MAX control (30 μl Jurkat M1 cell suspension+5 μl of HBSS, hepes 20 mM in 6% total DMSO+25 μl 5 μM carbacol) were used.

### Preparation of the Carbacol Solution

1 mg of carbacol was dissolved in 547.6 μl distilled water. 108 μL of this solution was mixed with 90 ml HBSS, hepes 20 mM, leading to a 12 μM carbacol solution.

### D4) Results

The fluorescence obtained in the CHO-NFAT-PAR2WT cells stimulated with increasing concentrations of SLIGRL-NH₂ (from 0.1 μM to 100 μM) is compared to that obtained in the CHO-NFAT-PAR-2Mut2 and CHO-NFAT-PAR2-Mut3 cells. The fluorescence measured with the CHO-NFAT-PAR-2Mut2 cells is higher than the fluorescence measured with CHO-PAR2-WT. The same observation is made with CHO-NFAT-PAR2-Mut3.

The CHO-NFAT-PAR2-MUT2 and the CHO-NFAT-PAR2-Mut3 cells, like the CHO-NFAT cells, are not activated by trypsin contrary to the CHO-NFAT-PAR2-WT which confirm that such cells possess no type of receptors containing a tethered ligand which might be activated by proteolytic clivage.

### E) Enzymatic Assay

An enzymatic test was performed to confirm that the compounds which have been:

positively screened according to the beta lactamase assays, and the assays described in D1) and D2); and

negatively screened according to the assay described in D3);

were not modulators of trypsine.

Experiment was performed according to the manufacturer indications (Roche kit CAT #108073). The assay was performed for 30 min at 37° C. using 250 ng trypsin (Sigma # T-7418), the OD was measured at 574 nm.

### F) Confirmation of an Agonist or Antagonist Effect of the Test Compounds

To determine if the compounds which have been:

positively screened according to the beta lactamase assays, and the assays described in D1) and D2); and

negatively screened according to the assays described in D3) and E);

had an agonist or an antagonist effect on the receptor, two tertiary tests were developped.

### F1) PAR-2 Activation, using SLIGRL 3 μM/CHO-NFAT-PAR2-MUT2

The day before the experiment, the cells were seeded into 384 well plate in a DMEM medium (50 μl) at 25000 cells/well;

The day of the experiment, the medium was discarded (by turning the plate), 50 μl of HBSS, hepes 20 mM were added to each well, then the buffer was discarded by turning the plate.

10 ml of Calcium kit loading solution (solution prepared as described above) and 10 ml HBSS, hepes 20 mM and 200 μl probenecid 250 μM were mixed, and 30 μl of this solution were added per well, plates were incubated during 2 hours at 37° C. 5% CO2.

The experiment took then place in two parts, as two reagents were added in rapid succession :

5 μl of the compound to be tested was first added to the cells using the FLIPR, and the fluorescence was measured during 90 seconds.

In the second part of the experiment, 25 μl of 7.2 μM SLIGRL was added with the FLIPR, and the fluorescence was measured again during 90 seconds.

### Effect of an Antagonist Compound

An antagonist compound has no effect on the fluorescence measured during the first part of the curve and prevents the increase the fluorescence measured during the second part of the experiment, compared to a curve measured with SLIGRL only.

### Effect of an Agonist Compound

An agonist compound leads to an increase of the fluorescence measured during the first part of the curve (due to a calcium release) and prevents the increase of the fluorescence measured during the second part of the experiment, due to the homologous desensitization of the receptor.

### Controls

A MIN control (CHO-NFAT-WT+30 μl calcium loading solution+5 μl of HBSS, hepes 20 mM in 6% DMSO, and addition of 25 μl 7.2 μM SLIGRL in the second part of the experiment) and a MAX control (CHO-NFAT-PAR2-Mut2+30 μl calcium loading solution+5 μl of HBSS, hepes 20 mM in 6% DMSO, and addition of 25 μl 7.2 μM SLIGRL in the second part of the experiment) were used.

### Preparation of the Compounds to be Tested

Products to be tested were diluted in a polypropylen 384 well plate with HBSS, Hepes 20 mM. The final concentration of DMSO should not be beyond 6% in the well.

### Preparation of the SLIGRL Solution (Neosystem réf: SP001568C)

1 mg SLIGRL was added to 8.355 ml HBSS, hepes 20 mM; then 107.68 ml HBSS, hepes 20 mM was added, leading to a SLIGR concentration of 7.2 μM.

### IC50 Calculations

During the second part of the experiment, we observed an inhibition of fluorescence measured compared to the fluorescence measured in absence of test compound. This was observed for an antagonist and also for an agonist.

An inhibition percentage was therefore calculated for each concentration of compound tested in this second part of the curve

[(UF antagonist/agonist)−(UF min)/(UF CHO-NFAT-PAR2-mut2)−(UF min)]×100

with:

UF antagonist/agonist: fluorescence units measured with the compound

UF min: fluorescence units measured with the CHO-NFAT-WT cells (=min)

UF CHO-NFAT-PAR2-mut2: fluorescence units measured with CHO-NFAT-PAR2-mut2 cells, without product (=max).

The curve (% inhibition=f(compound concentration)) was drawn and the concentration of compound leading to 50% inhibition was then deduced, representing the IC50.

### F2) PAR-2 Activation, using Trypsin 30 nM/CHO-NFAT-PAR2 -WT

The day before the experiment, the cells were seeded into 384 well plate in a DMEM medium (50 μl) at 25000 cells/well;

The day of the experiment, the medium was discarded (by turning the plate), 50 μl of HBSS, hepes 20 mM were added to each well, then the buffer was discarded by turning the plate.

10 ml of Calcium kit loading solution (solution prepared as described above) and 10 ml HBSS, hepes 20 mM and 200 μl probenecid 250 μM were mixed, and 30 μl of this solution were added per well, plates were incubated during 2hours at 37° C. 5% CO2.

The experiment took then place in two parts

5 μl of compound to be tested was first added to the cells with the FLIPR, and the fluorescence was measured during 90 seconds.

In the second part of the experiment, 25 μl of 72 nM trypsin was added with the FLIPR, and the fluorescence was measured again during 90 seconds.

### Effect of an Antagonist Compound

An antagonist has no effect on the fluorescence measured during the first part of the curve and prevents the increase of the fluorescence measured during the second part of the experiment, compared to a curve measured with trypsin only.

### Effect of an Agonist Compound

An agonist leads to an increase of the fluorescence measured during the first part of the curve (due to a calcium release) and prevents the increase of the fluorescence measured during the second part of the experiment due to the homologous desensitization of the receptor.

### Controls

A MIN control (CHO-NFAT-WT+30 μl calcium loading solution+5 μl of HBSS, hepes 20 mM in 6% DMSO, and addition of 25 μl 72 nM trypsin in the second part of the experiment) and a MAX control (CHO-NFAT-PAR2-WT+30 μl calcium loading solution+5 μl of HBSS, hepes 20 mM in 6% DMSO, and addition of 25 μl 72 nM trypsin in the second part of the experiment) were used.

### Preparation of the Compounds to be Tested

Products to be tested were diluted in a polypropylen 384 well plate with HBSS, Hepes 20 mM. The final concentration of DMSO should not be beyond 6% in the well.

### Preparation of the Trypsin Solution

1 mg trypsin was added to 41.6 ml HBSS, hepes 20 mM; then 7 ml of this solution was added to 90 ml HBSS, hepes 20 mM, leading to a trypsin concentration of 72 nM.

### IC50 Calculations

During the second part of the experiment, we observed an inhibition of fluorescence measured compared to the fluorescence measured in absence of test compound. This was observed for an antagonist and also for an agonist. An inhibition percentage was therefore calculated for each concentration of compound tested in this second part of the curve:

[(UF antagonist/agonist)−(UF min)/(UF CHO-NFAT-PAR2-mut2)−(UF min)]×100

with

UF antagonist/agonist: fluorescence units measured with the compound

UF min: fluorescence units measured with the CHO-NFAT-WT cells (=min)

UF CHO-NFAT-PAR2-mut2: fluorescence units measured with CHO-NFAT-PAR2-mut2 cells, without product (=max)

The curve (% inhibition=f(compound concentration)) was drawn and the concentration of compound leading to 50% inhibition was then deduced, representing the IC50.

### F3) Results

We have found several antagonist compounds with an IC50 of less than 10 μM.

Also, different agonist compounds with an IC50 of less than 0.2 μM were found.

### G) Binding Assays

### G1) Binding Assays using a Modified PAR-2

Cells grown to the point of about 85% confluence are dissociated in EDTA-containing saline, harvested by low-speed centrifugation, and resuspended at a concentration of about 3.5×10⁶ cells/ml in Earle's balanced buffer, pH 7.5, supplemented with 25 mM HEPES and 0.1% (w/v) BSA. Cell aliquots (0.2 ml final volume) are incubated in triplicate at 4° C. for 1 h along with approximately 10 6 cpm [3H]propionyl-tc-NH2 (approximately 3.5 nM) in either the absence or presence of increasing concentrations of unlabeled competing peptide. After incubation, reactions are terminated by rapid filtration of the binding reaction through Packard filterplate (GF/B) that have been treated with 0.5% PEI. The filterplates are washed twice with buffer and radioactivity retained on the filters is counted on a Packard Topcount. The amount of specific binding is calculated by substraction from the total amount of radioligand bound in absence of competing ligand, the amount of radioactivity bound in the presence of an excess (100 μM) of unlabeled tc-NH₂.

### G2) Binding Assays using a Modified PAR-1

A binding assay using a modified PAR-1 of the invention can be run according to the protocol described in Ahn et al., 1997 cited above.

### H) Selectivity Assays

Functional assays measuring the intracellular calcium increase are run after stimulation of PAR-1 or PAR-4. This allows to confirm that the modulators that were discovered are specific of PAR-2.

1. A modified PAR polypeptide, said modified polypeptide having an endogenous activating peptide made non functional and said modified PAR polypeptide being capable of interacting with an exogenous ligand for a wild-type PAR receptor, wherein said exogenous ligand has an ED50 for said modified PAR polypeptide significantly higher than its ED50 for a wild type PAR receptor.

2. The modified PAR polypeptide of claim 1, wherein said exogenous ligand has an ED50 for said modified PAR polypeptide at least 5, 10 or 50 times higher than its ED50 for a wild type PAR receptor.

3. The modified PAR polypeptide of claim 1, wherein a region extending from a first N-terminal residue of said PAR polypeptide up to a residue between the first N-terminal residue of the endogenous activating peptide and a last C-terminal residue of an amino-terminal extracellular domain of said PAR polypeptide is deleted.

4. The modified PAR polypeptide of claim 1, wherein said PAR receptor is selected from PAR-1, PAR-2, PAR-3 and PAR-4.

5. The modified PAR polypeptide of claim 4, wherein said modified polypeptide comprises a sequence selected from the group consisting of sequence SEQ ID NO:4, sequence SEQ ID NO:5, sequence SEQ ID NO:6, and functional variants and fragments thereof.

6. A polynucleotide encoding a polypeptide of claim 1.

7. The polynucleotide of claim 6, selected from:
(i) a polynucleotide encoding a polypeptide of SEQ ID NO: 4, 5 or 6;
(ii) a polynucleotide of sequence SEQ ID NO: 1, 2 or 3;
(iii) a polynucleotide hybridizing to a polynucleotide of (i) or (ii) and encoding a modified PAR polypeptide of claim 1;
(iv) a polynucleotide having at least 75% identity with a polynucleotide of (i), (ii) or (iii) and encoding a modified PAR polypeptide of claim 1; and
(v) a polynucleotide encoding a modified PAR polypeptide of claim 1 and a sequence of which differ from those of the polynucleotides (i), (ii), (iii) or (iv) due to the degeneracy of the genetic code.

8. The polynucleotide of claim 6, selected from:
(i) a polynucleotide encoding a polypeptide of SEQ ID NO: 4, 5 or 6;
(ii) a polynucleotide of sequence SEQ ID NO: 1, 2 or 3;
(iii) a polynucleotide hybridizing to a polynucleotide of (i) or (ii) and encoding a modified PAR polypeptide of claim 2;
(iv) a polynucleotide having at least 75% identity with a polynucleotide of (i), (ii) or (iii) and encoding a modified PAR polypeptide of claim 2; and
(v) a polynucleotide encoding a modified PAR polypeptide of claim 2 and a sequence of which differ from those of the polynucleotides (i), (ii), (iii) or (iv) due to the degeneracy of the genetic code.

9. The polynucleotide of claim 6, selected from:
(i) a polynucleotide encoding a polypeptide of SEQ ID NO: 4, 5 or 6;
(ii) a polynucleotide of sequence SEQ ID NO: 1, 2 or 3;
(iii) a polynucleotide hybridizing to a polynucleotide of (i) or (ii) and encoding a modified PAR polypeptide of claim 3;
(iv) a polynucleotide having at least 75% identity with a polynucleotide of (i), (ii) or (iii) and encoding a modified PAR polypeptide of claim 3; and
(v) a polynucleotide encoding a modified PAR polypeptide of claim 3 and a sequence of which differ from those of the polynucleotides (i), (ii), (iii) or (iv) due to the degeneracy of the genetic code.

10. The polynucleotide of claim 6, selected from:
(i) a polynucleotide encoding a polypeptide of SEQ ID NO: 4, 5 or 6;
(ii) a polynucleotide of sequence SEQ ID NO: 1, 2 or 3;
(iii) a polynucleotide hybridizing to a polynucleotide of (i) or (ii) and encoding a modified PAR polypeptide of claim 4;
(iv) a polynucleotide having at least 75% identity with a polynucleotide of (i), (ii) or (iii) and encoding a modified PAR polypeptide of claim 4; and
(v) a polynucleotide encoding a modified PAR polypeptide of claim 4 and a sequence of which differ from those of the polynucleotides (i), (ii), (iii) or (iv) due to the degeneracy of the genetic code.

11. The polynucleotide of claim 6, selected from:
(i) a polynucleotide encoding a polypeptide of SEQ ID NO: 4, 5 or 6;
(ii) a polynucleotide of sequence SEQ ID NO: 1, 2 or 3;
(iii) a polynucleotide hybridizing to a polynucleotide of (i) or (ii) and encoding a modified PAR polypeptide of claim 5;
(iv) a polynucleotide having at least 75% identity with a polynucleotide of (i), (ii) or (iii) and encoding a modified PAR polypeptide of claim 5; and
(v) a polynucleotide encoding a modified PAR polypeptide of claim 5 and a sequence of which differ from those of the polynucleotides (i), (ii), (iii) or
(iv) due to the degeneracy of the genetic code.

12. An expression vector comprising a polynucleotide of claim 6.

13. A recombinant host cell comprising a polynucleotide of claim 6.

14. A recombinant host cell comprising a vector of claim 12.

15. The recombinant host cell of claim 13 which does not express a wild-type PAR receptor.

16. The recombinant host cell of claim 14 which does not express a wild-type PAR receptor.

17. The recombinant host cell of claim 13, 14, 15 or 16 which expresses a reporter gene for detecting or measuring the activity of said modified PAR polypeptide.

18. A method of preparing a modified PAR polypeptide of claim 1, said method comprising the steps of:
a) obtaining a polynucleotide encoding said modified PAR polypeptide;
b) inserting said polynucleotide into an expression vector, said polynucleotide being functionally linked to a promoter; and
c) producing said modified PAR polypeptide from said polynucleotide.

19. A method of preparing a modified PAR polypeptide of claim 2, said method comprising the steps of:
a) obtaining a polynucleotide encoding said modified PAR polypeptide;
b) inserting said polynucleotide into an expression vector, said polynucleotide being functionally linked to a promoter; and
c) producing said modified PAR polypeptide from said polynucleotide.

20. A method of preparing a modified PAR polypeptide of claim 3, said method comprising the steps of:
a) obtaining a polynucleotide encoding said modified PAR polypeptide;
b) inserting said polynucleotide into an expression vector, said polynucleotide being functionally linked to a promoter; and
c) producing said modified PAR polypeptide from said polynucleotide.

21. A method of preparing a modified PAR polypeptide of claim 4, said method comprising the steps of:
a) obtaining a polynucleotide encoding said modified PAR polypeptide;
b) inserting said polynucleotide into an expression vector, said polynucleotide being functionally linked to a promoter; and
c) producing said modified PAR polypeptide from said polynucleotide.

22. A method of preparing a modified PAR polypeptide of claim 5, said method comprising the steps of:
a) obtaining a polynucleotide encoding said modified PAR polypeptide;
b) inserting said polynucleotide into an expression vector, said polynucleotide being functionally linked to a promoter; and
c) producing said modified PAR polypeptide from said polynucleotide.

23. A method of screening for, selecting or identifying a PAR activity modulator compound, said method comprising the steps of:
a) bringing a test compound into contact with a modified PAR polypeptide, said modified PAR polypeptide having an endogenous activating peptide which is made nonfunctional and said modified PAR polypeptide being capable of interacting with an exogenous ligand for a wild-type PAR receptor; and
b) selecting a compound which binds to said modified PAR polypeptide or determining the activity of said modified PAR polypeptide.

24. The method of claim 23, said method comprising the steps of:
a) bringing a test compound into contact with a modified PAR polypeptide, said modified polypeptide having an endogenous activating peptide made non functional, and said modified PAR polypeptide being capable of interacting with an exogenous ligand for a wild-type PAR receptor, wherein said exogenous ligand has an ED50 for said modified PAR polypeptide significantly higher than its ED50 for a wild type PAR receptor; and
b) selecting a compound which binds to said modified PAR polypeptide or determining the activity of said modified PAR polypeptide.

25. The method of claim 23, said method comprising the steps of:
a) bringing a test compound into contact with a modified PAR polypeptide, said modified polypeptide having an endogenous activating peptide made non functional, and said modified PAR polypeptide being capable of interacting with an exogenous ligand for a wild-type PAR receptor, wherein said exogenous ligand has an ED50 for said modified PAR polypeptide at least 5, 10 or 50 times higher than its ED50 for a wild type PAR receptor; and
b) selecting a compound which binds to said modified PAR polypeptide or determining the activity of said modified PAR polypeptide.

26. The method of claim 23, said method comprising the steps of:
a) bringing a test compound into contact with a modified PAR polypeptide, said modified polypeptide having an endogenous activating peptide made non functional, and said modified PAR polypeptide being capable of interacting with an exogenous ligand for a wild-type PAR receptor, wherein said exogenous ligand has an ED50 for said modified PAR polypeptide significantly higher than its ED50 for a wild type PAR receptor, and wherein a region extending from a first N-terminal residue of said PAR polypeptide up to a residue between the first N-terminal residue of the endogenous activating peptide and the last C-terminal residue of the amino-terminal extracellular domain is deleted; and
b) selecting a compound which binds to said modified PAR polypeptide or determining the activity of said modified PAR polypeptide.

27. The method of claim 23, said method comprising the steps of:
a) bringing a test compound into contact with a modified PAR polypeptide, said modified polypeptide having an endogenous activating peptide made non functional, and said modified PAR polypeptide being capable of interacting with an exogenous ligand for a wild-type PAR receptor, wherein said exogenous ligand has an ED50 for said modified PAR polypeptide significantly higher than its ED50 for a wild type PAR receptor, and wherein said PAR receptor is selected from PAR-1, PAR-2, PAR-3 and PAR-4; and
b) selecting a compound which binds to said modified PAR polypeptide or determining the activity of said modified PAR polypeptide.

28. The method of claim 23, said method comprising the steps of:
a) bringing a test compound into contact with a modified PAR polypeptide, said modified polypeptide having an endogenous activating peptide made non functional, and said modified PAR polypeptide being capable of interacting with an exogenous ligand for a wild-type PAR receptor, wherein said exogenous ligand has an ED50 for said modified PAR polypeptide significantly higher than its ED50 for a wild type PAR receptor, and wherein said modified polypeptide comprises a sequence selected from SEQ ID NO:4, sequence SEQ ID NO:5, sequence SEQ ID NO:6, and functional variants and fragments thereof; and
b) selecting a compound which binds to said modified PAR polypeptide or determining the activity of said modified PAR polypeptide.

29. The method of claim 24, 25, 26, 27 or 28, said method comprising the steps of:
a) bringing a test compound into contact with said modified PAR polypeptide; and
b) selecting a compound which binds to said modified PAR polypeptide.

30. The method of claim 24, 25, 26, 27 or 28, said method comprising the steps of:
a) bringing a test compound into contact with said modified PAR polypeptide; and
b) determining the activity of said modified PAR polypeptide.

31. The method of claim 30, wherein step b) further comprises the steps of:
a) providing a recombinant host cell expressing said modified PAR polypeptide in an appropriate culture medium;
b) adding a desired concentration of a test compound in said culture medium;
c) adding a reference ligand in said culture medium obtained at step b);
d) measuring the activity of said modified PAR polypeptide; and
e) comparing the activity of said modified PAR polypeptide obtained at step d) with the activity of said modified PAR polypeptide when step b) is omitted.

32. The method of claim 30 wherein said modified PAR polypeptide activity is determined through calcium release measurement.

33. The method of claim 31 wherein said modified PAR polypeptide activity is determined through calcium release measurement.

34. The method of claim 31, wherein said PAR polypeptide activity measure is direct.

35. The method of claim 30, said method further comprising the steps of:
a) providing a recombinant host cell coexpressing said modified PAR polypeptide and a reporter gene for detecting or measuring the activity of said modified PAR polypeptide;
b) adding a desired concentration of a test compound in said culture medium;
c) adding a reference ligand in said culture medium obtained at step b);
d) measuring reporter gene expression; and
e) comparing the reporter gene expression obtained at step d) with the reporter gene expression when step b) is omitted.

36. The method of claim 31 wherein the recombinant host cell consist of a CHO cell line.

37. The method of claim 35 wherein the recombinant host cell consist of a CHO cell line.

38. The method of claim 35 wherein the reporter gene is a beta lactamase gene.

39. The method of claim 36 wherein the reporter gene is a beta lactamase gene.

40. The method of claim 37 wherein the reporter gene is a beta lactamase gene.

41. The method of claim 38 wherein said betalactamase gene is placed under the control of a promoter comprising an NFAT domain sensitive to Ca⁺⁺ ions.

42. The method of claim 39 wherein said betalactamase gene is placed under the control of a promoter comprising an NFAT domain sensitive to Ca⁺⁺ ions.

43. The method of claim 40 wherein said betalactamase gene is placed under the control of a promoter comprising an NFAT domain sensitive to Ca⁺⁺ions.

44. The method of claim 23, 24,, 25, 26, 27 or 28 wherein the modified PAR polypeptide is a modified PAR-2 polypeptide.

45. The method according to claim 44, wherein the modified PAR polypeptide has a sequence selected from the group consisting of sequence SEQ ID NO:4, sequence SEQ ID NO:5, sequence SEQ ID NO:6, and functional variants and fragments thereof.

46. The method of claim 31 wherein the reference ligand is selected from the group consisting of SLIGRL, SLIGKV, SLIGR, propionyl-tc, trans-cinnamoyl-LIGRLO and SFLLR.

47. The method of claim 46 wherein the reference ligand is SLIGRL.

48. A kit for in vitro screening, selection or identifying of PAR activity modulator compounds, wherein said kit comprises:
a) a polypeptide as defined in claim 1, 2, 3, 4 or 5 or a recombinant host cell as defined in claim 13, 14, 15 or 16;
b) optionally, one or more reagents necessary to perform the PAR polypeptide activity measurement.


SEQUENCE LISTING (11 sequences)

SEQ ID NO: 1 - DNA, 1068 bp, Artificial
  Description of artificial sequence Mut2
gatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatgccaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 2 - DNA, 1026 bp, Artificial
  Description of artificial sequence Mut3
acagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatgccaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 3 - DNA, 963 bp, Artificial
  Description of artificial sequence Mut4
cttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatgccaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 4 - protein, 355 aa, Artificial
  Description of artificial sequence Mut2
DGTSHVTGKGVTVETVFSVDEFSASVLTGKLTTVFLPIVYTIVFVVGLPSNGMALWVFLFRTKKKHPAVIYMANLALADLLSVIWFPLKIAYHIHANNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 5 - protein, 341 aa, Artificial
  Description of artificial sequence Mut3
TVFSVDEFSASVLTGKLTTVFLPIVYTIVFVVGLPSNGMALWVFLFRTKKKHPAVIYMANLALADLLSVIWFPLKIAYHIHANNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 6 - protein, 320 aa, Artificial
  Description of artificial sequence Mut4
LPIVYTIVFVVGLPSNGMALWVFLFRTKKKHPAVIYMANLALADLLSVIWFPLKIAYHIHANNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 7 - DNA, 31 bp, Artificial
  Description of artificial sequence Oligonucleotide used for the construction of Mut2
aaggacgacgacgacggatcctctaaaggaa

SEQ ID NO: 8 - DNA, 31 bp, Artificial
  Description of artificial sequence Oligonucleotide used for the construction of    Mut2
ttcctttagaggatccgtcgtcgtcgtcctt

SEQ ID NO: 9 - DNA, 27 bp, Artificial
  Description of artificial sequence Oligonucleotide used for the construction of   Mut2
cgcggatccgatggcacatcccacgtc

SEQ ID NO: 10 - DNA, 27 bp, Artificial
  Description of artificial sequence Oligonucleotide used for the construction of Mut2 and Mut3
ggtcagccaaggccagattggccatgt

SEQ ID NO: 11 - DNA, 27 bp, Artificial
  Description of artificial sequence Oligonucleotide used for the construction of   Mut3
cgcggatccacagtcttttctgtggat